(19) United States
(12) Patent Application Publication
(10) Pub. No.: US 2026/0034072 A1
(43) Pub. Date: Feb. 5, 2026

(54) COATED NANOPARTICLES FOR THE TREATMENT OF PERIPHERAL ARTERY DISEASE

(71) Applicant: Board of Regents, The University of Texas System, Austin, TX (US)

(72) Inventors: Kytai T. NGUYEN, Mansfield, TX (US); Priyanka Raghunathan IYER, Arlington, TX (US); Tam Phung NGUYEN, Arlington, TX (US)

(73) Assignee: Board of Regents, The University of Texas System, Austin, TX (US)

(21) Appl. No.: 19/285,244

(22) Filed: Jul. 30, 2025

Related U.S. Application Data
(60) Provisional application No. 63/677,747, filed on Jul. 31, 2024.

(51) Int. Cl. A61K 9/51; A61K 9/50; A61K 38/18; A61P 9/10

(52) U.S. Cl. CPC A61K 9/5153; A61K 9/5068; A61K 9/5169; A61K 9/5192; A61K 38/1816; A61P 9/10

(57) ABSTRACT

Described herein are compositions and methods for treating peripheral artery diseases, including a composition comprising a nanoparticle defining an interior volume and an exterior surface; a payload disposed within the interior volume of the nanoparticle; and a cellular membrane coating disposed on or encapsulating the exterior surface of the nanoparticle; wherein the cellular membrane coating comprises an ICAM1 binding ligand.

### CROSS REFERENCE TO RELATED APPLICATIONS

This application claims priority pursuant to 35 U.S.C. § 119 to U.S. Provisional Patent Application No. 63/677,747, filed Jul. 31, 2024, which is hereby incorporated by reference in its entirety.

### FIELD

The present application relates to the field of engineered cellular membrane-coated nanoparticles comprising a therapeutic agent and methods of treating peripheral artery disease using such nanoparticles.

### BACKGROUND

Peripheral arterial disease (PAD) is a narrowing of the peripheral arteries that carries blood away from the heart to other parts of the body. The most common type is lower-extremity PAD, in which blood flow is reduced to the legs and feet. According to the National Heart, Lung, and Blood Institute (NHLBI), plaques may reduce or fully block the flow of oxygen-rich blood through arteries to the body's vital organs and the limbs. The blockage of an artery by a plaque causes more than 50% reduction in the blood flow when compared to normal artery. Reduced blood flow in the lower extremities often hinders the patient's ability to walk. PAD globally affects more than 235 million people with a mortality rate exceeding 50% for patients suffering from Critical Limb Ischemia—an extreme case of PAD. PAD affects more than eight million individuals only in the United States, resulting in higher morbidity and mortality.

Diagnosing PAD is done by evaluation of ankle: arm blood pressure index, MRI, angiogram, or blood test. According to CDC, the most common symptom of PAD is pain in the legs with physical activity, such as walking, that gets better after rest. PAD patients are under risk of compounding other cardiovascular diseases and limb related morbidity. Occurrence in the elderly (>80 years of age) increases by 20%. However, 50% of PAD are asymptomatic, which often causes delays in treatment. The prevalence of PAD in men and women is very similar; however, the occurrence increases in older men compared to older women.

Current treatments include angioplasty, drug eluting stent, autologous/syn thetic graft, growth factor delivery, and cell-based therapies. Surgical interventions for PAD, including angioplasty, synthetic grafts, and drug-eluting stents, provide the immediate benefit of restoring blood perfusion. However, these procedures are invasive and pose risks, including but not limited to restenosis, thrombosis, low delivery of drug, and graft unavailability.

Notwithstanding progress made in surgical and non-surgical treatment of PAD, new methods of therapy are needed. As disclosed herein, nanoparticles have been developed that mimic the extracellular matrix via a cellular membrane coating engineered to express binding ligands and deliver pro-angiogenic therapeutic factors to stimulate the formation of new blood vessels. By promoting angiogenesis, these nanoparticles enhance blood flow to ischemic tissues, alleviate symptoms, and improve tissue perfusion in patients with PAD.

### SUMMARY

In one aspect, the present application discloses a composition comprising: a nanoparticle defining an interior volume and an exterior surface; a therapeutic agent disposed within the interior volume of the nanoparticle; and a cellular membrane coating disposed on or encapsulating the exterior surface of the nanoparticle; wherein the cellular membrane coating comprises an Intercellular Adhesion Molecule-1 alpha receptor (ICAM1 or ICAM-1) binding ligand. In some embodiments, the nanoparticle is a polymeric nanoparticle, an inorganic nanoparticle or a lipid-based nanoparticle. In some embodiments, the nanoparticle is a polymeric nanoparticle and comprises poly(lactic-co-glycolic acid), polylactic acid, polyglycolic acid, polyglutamic acid, polycaprolactone, polylysine or mixtures thereof. In some embodiments, the therapeutic agent comprises a small molecule therapeutic, a regenerative factor, a protein peptide and/or a plasmid for gene therapy or combinations thereof. In some embodiments, the therapeutic agent comprises vascular endothelial growth factor, fibroblast growth factor, hepatocyte growth factor, stromal derived growth factor, platelet derived growth factor, and/or erythropoietin. In some embodiments, the therapeutic agent comprises peripheral blood-derived or bone marrow-derived stem cells, mesenchymal stem cells, or marker-specific subsets of bone marrow cells with angiogenic properties, or the therapeutic agent comprises peptides, antibodies, and/or aptamers that recognize specific targets associated with PAD pathology. In some embodiments, the cellular membrane is derived from a pericyte. In some embodiments, the pericyte cellular membrane is engineered to express the ICAM1 binding ligand. In some embodiments, the ICAM1 binding ligand comprises an integrin, fibrinogen, or hyaluronan, or a binding fragment of any of the foregoing. In some embodiments, the ICAM1 binding ligand comprises LFA-1 or Mac-1, or a binding fragment of any of the foregoing.

In one aspect, the present application discloses a pharmaceutical composition comprising an effective amount of any membrane-coated nanoparticle composition disclosed herein and a pharmaceutically acceptable carrier or excipient. In some embodiments, the nanoparticle is a polymeric nanoparticle. In some embodiments, the cellular membrane is derived from a pericyte. In some embodiments, the ICAM1 binding ligand is an integrin, fibrinogen, or hyaluronan, or a binding fragment of any of the foregoing. In some embodiments, the ICAM1 binding ligand comprises LFA-1 or Mac-1, or a binding fragment of any of the foregoing.

In another aspect the present application discloses a method of treating a condition of a patient in need thereof, the method comprising: disposing any membrane-coated nanoparticle composition disclosed herein within a biological compartment of the patient. In some embodiments, the condition is peripheral artery disease. In some embodiments, the nanoparticle is a polymeric nanoparticle. In some embodiments, the cellular membrane is derived from a pericyte. In some embodiments, the therapeutic agent is erythropoietin. In some embodiments, the nanoparticle releases EPO upon degradation of the cellular membrane coating and polymer comprising the nanoparticle. In some embodiments, the ICAM1 binding ligand is an integrin, fibrinogen, or hyaluronan, or a binding fragment of any of the foregoing.

In yet another aspect, the present application discloses a method of manufacturing a membrane-coated nanoparticle, wherein a therapeutic agent is disposed within the interior volume of the nanoparticle; a cellular membrane coating extracted from a pericyte is disposed on or encapsulates the exterior surface of the nanoparticle; and the nanoparticle comprises a polymer; the method comprising obtaining a nanoparticle comprising a polymer; loading the nanoparticle with a therapeutic agent; coating a cellular membrane on the nanoparticle, wherein the cellular membrane comprises an ICAM1 binding ligand. In some embodiments, the method further comprises engineering a pericyte to express an ICAM1 binding ligand on the cell membrane and extracting the engineered cellular membrane comprising an ICAM1 binding ligand.

### BRIEF DESCRIPTION OF THE DRAWINGS

FIG. 1 is a cartoon of the plasmid construct of the ICAM1 binding ligand, in one embodiment of the present application, where the insert sequence in the plasmid encodes an ICAM1 binding ligand.

FIG. 2 is a representation of a gel electrophoresis of digested and undigested ICAM1 ligand plasmid, showing the ICAM1 binding ligand band at about 2500 bp which is the size of the ICAM1 binding ligand gene inserted.

FIG. 3A is a representation of a transmission electron microscopy image of PM-DNPs (erythropoietin (EPO)-loaded poly(lactic-co-glycolic acid) (PLGA)-nanoparticles (NPs) coated with normal (non-engineered, not expressing ICAM1 binding ligand) pericyte membranes) displaying the membrane coating compared to uncoated PLGA-EPO (DNPs), showing that both exhibited spherical shape.

FIG. 3B is a graph of drug release data of PLGA-EPO (DNPs) and PM-DNPs, showing a release of about 60%.

FIG. 3C is a graph of particle sizes of TPM-DNPs (EPO protein-loaded PLGA NPs coated with the pericyte membranes expressing ICAM1 binding ligand), showing stability in each of serum, human simulated body fluid (SBF), NaCl and water.

FIG. 4 is a graph of a resonant shift using ResoSens label-free optical detection, representing the binding kinetics of TPM-DNPs (transfected), PM-DNPs (non-transfected), anti-ICAM1 antibody (control).

FIG. 5A is a graph of the uptake of TPM-DNP, 3T3-DNPs (Fibroblast membrane-coated NPs, PM-DNPs, and DNPs by mouse endothelial cells (ECs) at a nanoparticle concentration of 0 to 500 g/mL.

FIG. 5B is a graph of the uptake of PM-DNPs and TPM-DNPs by activated and non-activated ECs.

FIG. 6A is a graph of the percent cell viability of TPM-DNPs on ECs treated with TNF-α to stimulate an inflammatory condition to determine the nanoparticle in vitro cytotoxic effect at different concentrations (0 μg/mL-1000 μg/mL).

FIG. 6B is a graph of the percent cell viability after administration of free erythropoietin (EPO), free VEGF, DNPs, PM-DNPs and TPM-DNPs and viability on Day 1, 3 and 5. No treatment (cells in low serum media) served as a negative control.

FIG. 7A is a graph of percent wound closure, cell migration of activated ECs in 2% serum (n=3), treated with free EPO, free VEGF, DNPs, PM-DNPs and TPM-DNPs.

FIG. 7B is a graph of percent wound closure, cell migration of activated ECs in low serum media were seeded on matrigel and treated with free EPO, free VEGF, DNPs, PM-DNPs and TPM-DNPs for 12 hours (n=3).

FIG. 8A is a graphical representation of the percentage reduction of blood perfusion over time of an ischemic leg compared to a healthy leg, showing the significant reduction of blood flow in a PAD leg, where Day 0 is before surgery.

FIG. 8B is a bar graph of the distance traveled on a treadmill, using a PAD model, comparing healthy and ischemia induced mice.

FIG. 9A is a bar graph of nanoparticle uptake by mouse organs following intramuscular (IM) injection of TPM-DNPs and PM-DNPs, each labeled with Cy5 florescence.

FIG. 9B is a bar graph of nanoparticle uptake by mouse organs following intravenous (IV) injection of TPM-DNPs and PM-DNPs, each labeled with Cy5 florescence.

FIG. 10A is a graph of blood perfusion improvement (%) over days 0-20, representing a time course of the ischemic-to-control leg blood flow ratio in the hindlimb of a mouse in a PAD model. The blood flow of the ischemic hindlimb is expressed as the ratio between the perfusion of the ischemic limb and the uninjured limb. As shown, TPM-DNPs statistically increased blood perfusion when compared to Saline, Free EPO, and TPM-blank NPs.

FIG. 10B is a bar graph of limb physical improvement (m) comparing TPM-DNPs, Free EPO, TPM-blank NPs and Saline in mice, showing TPM-DNPs resulted in greater endurance compared to the other groups.

FIG. 11 is a graphical representation of the percentage of cells expressing each of CD31, CD34, EPOR and Ki67 per field of view microscope, based on an analysis of immunohistochemistry staining of ICAM1 biomarkers on treated PAD mice, comparing TMP-DNPs and saline (vehicle), showing that TMP-DNP significantly decreased the number of cells positive with ICAM1.

### DETAILED DESCRIPTION

Embodiments described herein can be understood more readily by reference to the following detailed description, examples, claims, and attached Appendix. Elements, apparatus and methods described herein, however, are not limited to the specific embodiments presented in the detailed description, examples, claims, and attached Appendix. In particular, these embodiments are merely illustrative of the principles of the present invention. Accordingly, this disclosure is not intended to embrace all such alternatives, modifications and variations that fall within the spirit and broad scope of the specification and in view of the claims.

All publications, patents and patent applications mentioned in this specification are herein incorporated in their entirety by reference into the specification, to the same extent as if each individual publication, patent, or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9. When a range of integers is given, the range includes any number falling within the range and the numbers defining ends of the range. For example, when the terms “integer from 1 to 20” is used, the integers included in the range are 1, 2, 3, 4, 5, 6, 7, 8, 9, 10, etc., up to and including 20. All ranges disclosed herein are also to be considered to include the end points of the range, unless expressly stated otherwise. For example, a range of “between 5 and 10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

Furthermore, the terms “substantially,” “approximately,” and “about,” as used herein when referring to a measurable value such as an amount of a composition of this application, dose, time, temperature, and the like, is meant to encompass variations of ±20%, ±10%, ±5%, ±1%, ±0.5%, or even ±0.1% of the specified amount. The term “consists essentially of” (and grammatical variants) shall be given its ordinary meaning and shall also mean that the composition or method referred to can contain additional components as long as the additional components do not materially alter the composition or method. The term “consists of” (and grammatical variants) shall be given its ordinary meaning and shall also mean that the composition or method referred to is closed to additional components. The term “comprising” (and grammatical variants) shall be given its ordinary meaning and shall also mean that the composition or method referred to is open to contain additional components.

It is also to be understood that the article “a” or “an” refers to “at least one,” unless the context of a particular use requires otherwise.

Also as used herein, “and/or” refers broadly to and encompasses any and all possible combinations of one or more of the associated listed items, as well as the lack of combinations when interpreted in the alternative (“or”).

Pharmaceutical compositions and methods of preparation and uses thereof are disclosed. Unless otherwise defined, all technical and scientific terms used herein have the same meaning as commonly understood by one of ordinary skill in the art to which this subject matter belongs. The terminology used in the description of the subject matter herein is for the purpose of describing particular embodiments only and is not intended to be limiting of the subject matter. The present disclosure will be better understood with reference to the following definitions.

The term “effective amount,” as used herein, refers broadly to that amount of a recited therapeutic or composition effective to treat, prevent, or reduce the severity or progression of a disorder in a subject, such as a human subject. This includes improving the subject's condition (e.g., in one or more symptoms), delaying or reducing the progression of the disease and/or disorder, preventing or delaying the onset of the disorder, and/or changing clinical parameters, disease or illness, etc., as would be well known in the art.

For example, an effective amount can refer to the amount of a composition or therapeutic that improves a condition in a subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

As used herein, the terms “treating,” “treatment,” and the like are used to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder, or the relief or elimination of a symptom thereof. Thus, treatment includes preventing or protecting against the disease or disorder, that is, causing the clinical symptoms not to develop; and/or inhibiting the disease or disorder, that is, arresting or suppressing the development of clinical symptoms; and/or relieving the disease or disorder that is, causing the regression of clinical symptoms; and/or reducing the metastasis of the primary tumor or cancer.

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, horses, cats, dog, rabbits, rodents (e.g., rats or mice), monkeys, etc. Human subjects include neonates, infants, juveniles, adults and geriatric subjects. The subject “in need of” the methods disclosed herein can be a subject that is experiencing a disease state and/or is anticipated to experience a disease state, and the methods and compositions of the present application are used for therapeutic and/or prophylactic treatment.

As used herein, “DNPs” refer to erythropoietin-loaded PLGA nanoparticles without any cell membrane coating.

As used herein, “TPM-DNPs” refer to erythropoietin protein-loaded PLGA nanoparticles coated with pericyte membranes expressing an ICAM1 binding ligand.

As used herein, “PM-DNPs” refer to erythropoietin-loaded PLGA nanoparticles coated with normal (non-engineered, not expressing ICAM1 binding ligand) pericyte membranes.

As used herein “cellular membrane” refers to a membrane enclosing or separating structure acting as a selective barrier, within or around a cell, wherein the cell is typically a pericyte. The cellular membrane is selectively permeable to ions and organic molecules and controls the movement of substances in and out of cells. The cellular membrane comprises a phospholipid uni- or bilayer, and optionally associated proteins and carbohydrates. As used herein, the cellular membrane refers to a membrane obtained from a naturally occurring biological membrane of a cell or one derived from a cell, most typically a pericyte. As used herein, the term “naturally occurring” refers to one existing in nature. As used herein, the term “derived from” refers to any modification of the natural membrane, such as isolating the cellular membrane, creating portions or fragments of the membrane, removing and/or adding certain components, such as lipid, protein or carbohydrates, from or into the membrane taken from a cell, such as a pericyte. A membrane can be derived from a naturally occurring membrane by any suitable methods. For example, a membrane can be prepared or isolated from a cell and the prepared or isolated membrane can be combined with other substances or materials to form a derived membrane. In another example, a cell can be engineered to produce binding ligands that are incorporated into its membrane in vivo, and the cellular membrane can be prepared or isolated from the cell to form a derived or engineered cellular membrane. In some embodiments, a cellular membrane is engineered to produce an ICAM1 binding ligand. In some embodiments, a pericyte cellular membrane is engineered to produce an ICAM1 binding ligand

Peripheral arterial disease (PAD) is a health condition that poses significant challenges due to arterial occlusions in the lower extremities, leading to acute ischemia and high morbidity and mortality rates

As used herein, “nanoparticle” or “NP” refers to nanostructure, particles, vesicles, or fragments thereof having at least one dimension (e.g., height, length, width, or diameter) of between about 1 nm and about 10 μm. The nanoparticles disclosed herein can be composed of organic materials or other materials and can alternatively be implemented with porous particles; most typically the nanoparticles are composed of polymer. As used herein, the nanoparticle has an inner core covered by an outer surface comprising the membrane as disclosed herein.

Nanoparticles, by encapsulating a therapeutic, can maintain the therapeutic intact thereby avoiding degradation, increasing circulation time, and improving targeting delivery, such as via surface modification with an engineered cellular membrane as disclosed herein. As further disclosed herein, nanoparticles can encapsulate a variety of therapeutic agents including but not limited to stem cell-based therapies, proteins, such as growth factors and angiogenic factors, gene therapy and/or anti-inflammatory drugs.

Modification of nanoparticle surfaces with targeting moieties, such as ICAM1 binding ligands, enhances the ability of the NP to bind to cells of interest. Such specific ligand-receptor interactions can facilitate the uptake of nanoparticles inside cytoplasm via receptor-mediated endocytosis. The nanoparticles can then be delivered directly to the site of vascular lesions, allowing for targeted and controlled release of the therapeutic cargo. This targeted drug delivery approach minimizes systemic side effects and enhances the efficacy of the treatment.

Non-surgical approaches such as protein delivery, gene delivery, and stem cell-based therapy take advantage of gradually enhancing angiogenesis without serious complications. Cell based therapy, such as stem cell-based therapies, rely on the transplantation of exogenous cells to the site of action to promote tissue regeneration and angiogenesis. Incorporating such agents within the surface modified nanoparticles disclosed herein enables their robust delivery to the site of action.

As disclosed herein, cell membrane-coated drug carriers provide unique drug delivery features and functions. Pericyte cell membranes show advantages related to improved drug delivery efficacy, better targeting of injured endothelial cells via camouflaging, and minimizing unwanted interactions with reticuloendothelial and renal system components. Coating nanoparticles with pericyte membranes expressing high levels of adhesion molecules, such as ICAM1 binding ligand, enable them to reach and localize at the targeted tissues, such as ischemic sites of interest in the treatment of PAD. Coating cell membranes on the surface of the nanoparticle as disclosed herein increases the specific accumulation and facilitates trafficking and interactions to targeted injured muscle tissues via native stem cell membrane adhesion molecules.

The presence of the engineered cell membrane coating provides NPs with a stealth-like effect, extending their circulation time in the bloodstream by evading recognition and clearance by the reticuloendothelial system. This prolonged circulation enhances their ability to accumulate at the site of vascular lesions and exert therapeutic effects over an extended period. Overall, engineered cell membrane-coated NPs represent a promising strategy for the targeted delivery of therapeutics in PAD treatment. Their biomimetic nature, biological compatibility and targeted drug delivery capabilities make them an attractive platform for further development and clinical translation in the management of PAD.

Newly formed blood vessels consist primarily of two types of cells: endothelial cells (ECs), which line the vessel walls, and pericytes, which surround and support the vascular tube's surface. The process of revascularization, aimed at enhancing blood flow in ischemic tissues, involves the proliferation and migration of endothelial cells, along with the recruitment of pericytes to form stable collateral vessels that improve tissue perfusion.

Interactions of endothelial cells and pericytes in the blood vessel are essential for regulation of vascular formation, stabilization, remodeling, and functioning. Insufficient pericyte coverage of blood vessels are often associated with cardiovascular diseases. Pericyte loss from the vessel wall or their dysfunction due to ischemia leads to the progression of many pathological conditions. Interactions between pericytes and endothelial cells also include ligand-receptor mediated cellular signaling pathways and play an important role in angiogenesis. Pericytes have been shown to exhibit myogenic potential and without being bound by theory may be involved in the modulation of inflammation through the production of paracrine molecules. Pericyte loss from the vessel wall or their dysfunction, including dysfunction due to ischemia, can lead to the progression of many pathological conditions. Pericytes, endothelial progenitor cells, and mesenchymal cells have all been successful in rescuing limb ischemia by inducing angiogenesis. Pericyte coverage to the ECs is often disrupted during the ischemic condition, resulting in poor capillary permeability and defective angiogenesis.

Current pro-angiogenic strategies, including delivery of growth factors and growth factor-encoding genes, have proven beneficial effects for treating PAD in preclinical studies, Such therapeutics, for example VEGF, FGF, IGF, and PDGF, as well as gene therapy of VEGF, HGF, FGF, HIF-la, and SDF-1 can be included within the membrane-coated nanoparticles disclosed herein. Inclusion in nanoparticles disclosed herein overcomes a number of limitations, including low therapeutic effects and limited protein availability over time, mostly due to a short half-life and enzymatic degradation, off-target deposition, found with systemic delivery, as well as ineffectiveness in ischemic tissues.

The ICAM1 binding ligand expressed pericyte membrane-coated NPs disclosed herein, including those loaded with a therapeutic agent, such as EPO, target injured/ischemic tissues, mimic the interactions between pericytes and endothelial cells to improve the vessel maturation, mediate capture of EPCs (Endothelial Progenitor Cells) to provide targeted and controlled release of therapeutic agent to the targeted cells, and facilitate tissue regeneration, including treating PAD. The hybrid biofunctionalized and biomimetic cell membrane system of the present application is a universal drug carrier that can load any therapeutic agent.

Cell-based therapy and nanotechnology are paired herein to effectively target regions of ischemia while protecting endothelial/vascular cells and facilitating neovascularization under stress conditions. The new cell membrane coated biomimetic nanoparticle platforms with hybrid functionalities overcomes existing limitations of a number of current treatments, including those for PAD.

As disclosed herein, TPM-DNPs are developed and characterized, in particular as ischemic-targeted drug carriers. EPO protein-loaded PLGA NPs coated with the ICAM1 binding ligand expressing pericyte membranes were fabricated.

The process of engineered cell membrane-based therapy generally begins with the isolation of cell membranes from donor cells or engineered cells (e.g., pericytes). These membranes are isolated through a hypotonic solution or homogenization process. The isolated engineered membranes are coated onto the surface of nanoparticles, prepared from, for example PLGA, through extrusion or conjugation process. The PLGA-NPs coated with engineered cell membranes are loaded with desired therapeutic agents, such as for example, EPO. The resulting compositions are delivered to target cells through fusion with the cell membrane.

TPM-DNPs, PM-DNPs and DNPs using standard emulsion and extrusion methods were constructed, and the stability properties of these respective NPs in relevant media were evaluated. NPs were characterized for their physical-chemical properties such as particle sizes and drug loading efficacies. NPs were also incorporated with a tracer agent for in vitro and in vivo imaging studies.

As further disclosed herein, the targeting and therapeutic properties of TPM-DNPs were evaluated in vitro. Nanoparticle suspensions at different concentrations were evaluated for cytotoxicity to endothelial cells under stress conditions using cell survival assays. The therapeutic efficacy of the erythropoietin protein-loaded TPM-DNP was evaluated for cell proliferation, tube formation (angiogenesis), migratory ability, toxicity, hemo- and cyto-compatibility. The membrane coated nanoparticles loaded with EPO were also evaluated for ability to target cells under static and flow conditions (cell uptake of these nanoparticles in the inflammation induced endothelial cells).

As additionally disclosed herein, the in vivo properties of TPM-DNPs were studied. In particular, TPM-DNPs were investigated in vivo using hindlimb ischemia animal (mice) models for PAD treatment. A comparative study analysis for imtramuscular (IM) vs. intravenous (IV) was performed for analyzing better drug delivery strategy using dye-loaded NPs. The efficacy of the entire system was further evaluated in terms of increase in walking ability, blood perfusion, and neovascularization (immunostaining).

Pericytes exert an important role on endothelial cell proliferation, migration, and stabilization. Endothelial cells, in turn, stimulate expansion and activation of the pericyte precursor cell population. The detachment of pericytes from endothelium under pathological conditions leads to pericyte deficiency at the microvascular interstitial interface, resulting in unstable microvasculature and hence vessel rarefaction. Interactions between endothelial cells and pericytes in the blood vessel wall play a major role in the regulation of vascular formation, stabilization, remodeling, and function. To benefit from the full function of pericytes for angiogenic benefits, a bioengineered and functionalized pericyte membrane coated NPs was prepared according to the methods disclosed herein. Pericyte membrane coated NPs are used as gene/protein carriers to provide targeting to the injured/ischemic muscles and enable sustained release of therapeutic agents, leading to significant advances in the treatment of PAD.

NPs coated with pericyte membranes engineered with ICAM1 binding ligand are shown herein to target ischemic tissue through upregulation of ICAM1 expression in injured ECs and establish pericyte-EC interactions as well as recruit and home EPCs in ischemic tissues. TPM-DNPs not only served as carriers to deliver therapeutic agents to enhance the growth of blood vessels but also stimulated EPC-derived blood vessel formation and induced neovascularization and established pericyte-EC interactions required for stabilizing the vasculature. The interaction between stem cells like pericytes is not well understood in the field of cardiovascular diseases, but pericytes have been shown to regulate and stabilize angiogenesis in remodeling environments.

Although EPO has been shown to participate in pro-angiogenic processes, vascular integrity maintenance, and EC survival under hypoxic conditions, there have been few attempts to apply these factors to PAD treatments. EPO released from gelatin microspheres and fibrin gels has been shown to increase capillary densities and blood flow compared to un-encapsulated EPO in ischemic animal models. Without being bound by theory, since EPO is involved in stimulating EPC mobilization, providing cell protection/survival, and facilitating angiogenesis under hypoxia, delivery of EPO via TPM-DNPs facilitates recruitment of EPCs and further induces EPC proliferation, protection/survival, and subsequent enhance angiogenesis. EPO is an effective model therapeutic protein loaded in the membrane-coated nanoparticle system of the present application. TPM-DNPs facilitate targeted drug delivery by encapsulating therapeutic agents and directing them to specific cells, such as cancer or immune cells, through receptor-mediated interactions. This design enhances cellular uptake, enabling efficient internalization via endocytosis or membrane fusion. Once inside the cell, TPM-DNPs release the EPO protein in a controlled manner. Through these mechanisms, TPM-DNPs improve the therapeutic efficacy of treatment while reducing systemic toxicity and side effects and help in neovascularization.

Described herein are novel engineered pericyte membrane-coated nanoparticles and/or nanocarriers for targeted drug delivery to treat PAD. In some embodiments, these nanoparticles (TPM-DNPs) have a core made of poly(lactic-co-glycolic acid) (PLGA) loaded with EPO. In some cases, the core is further coated with membranes of cells engineered to express ligands binding with Intercellular Adhesion Molecule-1 alpha receptor (ICAM1 or ICAM-1) to specifically achieve targeting and the local delivery of payloads, such as therapeutic agents, and to establish endothelial cell (EC)-pericyte interactions at diseased sites, thereby promoting stemness, cell protection, proliferation, and angiogenesis for PAD treatment.

As disclosed herein, the nanoparticles coated with natural cellular membrane components, such as derived from a pericyte cell, facilitates stealth characteristics, particularly by reducing clearance by the mononuclear phagocyte system. A pericyte-derived membrane coating as disclosed herein demonstrates improved stability under physiological conditions. The absence of exogenous proteins in the pericyte-derived membrane coating minimizes immune activation when administered to a patient in need of therapy.

The pericyte-derived membranes of the present application in one embodiment are engineered to express molecules binding with ICAM1 leveraging native homing properties, thereby enhancing accumulation at disease sites without additional targeting ligands. In another embodiment, the pericyte-derived membranes of the present application are further engineered, such that the membranes coated on the nanoparticles present one or more ligands or receptor motifs, enabling broader and/or more selective targeting.

In some variations of the compositions disclosed herein, protective reagents can be included, such as BSA, dextrose, glucose, and/or glycerol.

As disclosed herein, the extracted membrane from the engineered pericyte cells is often in the form of a micellar structure. The phospholipid bilayers could be on the inside or the outside part of the membrane coated nanoparticles. When the ICAM1 binding ligand is facing the inner side of the membrane coated NPs, the ligand would generally not be visible on the outside of the NPs, and therefore might bind less effectively to ICAM1 present at the injured endothelium. Further during the extrusion process, inside-out or outside-in membrane molecules may possibly coat onto PLGA NPs, which may reduce the targeting moiety on the nanoparticle surfaces, leading to lower targeting efficacy. Thus in some embodiments disclosed herein, any of double-sided, intracellular-facing and extracellular facing ICAM1 binding ligands are incorporated.

In alternate methods for the preparation of nanoparticle formulation, precipitation can be used to prepare the nanoparticles without adding PVA or any surfactant.

In some embodiments, an antibody targeting endothelial cells, in addition to the ICAM1 binding ligand, can additionally be conjugated and/or expressed to target PAD.

Section I. Therapeutic-Loaded PLGA NPs Coated with Pericyte Membrane Expressing ICAM1 Binding Ligand

The membrane-coated nanoparticles disclosed herein generally have a diameter from about 10 nm to about 10 μm. In certain embodiments, the diameter of the coated nanoparticle is about 150 nm to about 500 nm. In other embodiments, the diameter of the coated nanoparticle can be about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, and 10 μm, or any suitable subranges within the about 10 nm to about 10 μm range, e.g., a diameter from about 150 nm to about 300 nm.

The present application further provides in some embodiments a membrane-coated nanoparticle substantially lacking constituents of the cell from which the cellular membrane is derived. For example, the present nanoparticle can lack, in terms of types and/or quantities, at least 10%, 20%, 30%, 40%, 50%, 60%, 70%, 80%, 90%, 95%, 96%, 97%, 98%, 99% or 100% of the constituents of the pericyte from which the cellular membrane is derived. In certain embodiments, the nanoparticle of the present application substantially lacks cytoplasm, nucleus and/or cellular organelles of the cell from which the cellular membrane is derived.

In other embodiments, the nanoparticle disclosed herein substantially maintains natural structural integrity or activity of the cellular membrane or the constituents of the cellular membrane. The activity of the cellular membrane can include, but is not limited to, binding activity, receptor activity, signaling pathway activity, and any other activities a normal naturally occurring cellular membrane or the constituents of the cellular membrane would have in a pericyte. In certain embodiments, the nanoparticle disclosed herein is biocompatible and/or biodegradable. For example, the nanoparticle can maintain, in terms of types and/or quantities, at least 10%, 20%, 30%, 40%, 50%, 60%, 70%, 80%, 90%, 95%, 96%, 97%, 98%, 99% or 100% of the natural structural integrity or activity of the cellular membrane or the constituents of the cellular membrane, such as the cellular membrane of a pericyte.

In one aspect, the present application discloses a composition comprising: a nanoparticle defining an interior volume and an exterior surface; a therapeutic agent disposed within the interior volume of the nanoparticle; and a cellular membrane coating disposed on or encapsulating the exterior surface of the nanoparticle; wherein the cellular membrane coating comprises an ICAM1 binding ligand.

In some embodiments, the nanoparticle is a polymeric nanoparticle, an inorganic nanoparticle or a lipid-based nanoparticle. Such nanoparticles and their synthesis is well-known in the art, and can be found generally in, for example, Mitchell, Michael J., et al. “Engineering precision nanoparticles for drug delivery.” Nature reviews drug discovery 20.2 (2021): 101-124. In some embodiments, the nanoparticle is a polymeric nanoparticle. Exemplary synthesis of polymeric nanoparticles can be found, for example, in Beach, M. A., et al. (2024). Polymeric nanoparticles for drug delivery. Chemical Reviews, 124 (9), 5505-5616. In some embodiments, the polymer nanoparticle comprises a dendrimer, a polymeric micelle, a polymersome or a nanosphere. In some embodiments, the polymeric nanoparticle comprises poly(lactic-co-glycolic acid), polylactic acid, polyglycolic acid, polyglutamic acid, polycaprolactone, polylysine or mixtures thereof. In some embodiments, the nanoparticle is an inorganic nanoparticle, such as for example, a quantum dot. In some embodiments, the inorganic nanoparticle comprises silica, iron oxide or gold. Synthesis of such inorganic particles can be found, for example in Cruz, J. C., & Reyes, L. H. Inorganic Nanocarriers: Gold Nanoparticles, Iron Nanoparticles, Silica Nanoparticles, Quantum Dots. In Nanocarriers for Nucleic Acids and Proteins (pp. 105-140). CRC Press. In some embodiments, the nanoparticle is a lipid-based nanoparticle. In some instances, the lipid-based nanoparticle is a noisome, a liposome, a lipid nanoparticle or an emulsion. The synthesis of such nanoparticles can be found, for example, in Amoabediny, Ghasem, et al. “Overview of preparation methods of polymeric and lipid-based (niosome, solid lipid, liposome) nanoparticles: A comprehensive review.” International Journal of Polymeric Materials and Polymeric Biomaterials 67.6 (2018): 383-400.

In some embodiments, the therapeutic agent comprises a small molecule therapeutic, a regenerative factor, a protein peptide and/or a plasmid for gene therapy or combinations thereof. In some embodiments, the therapeutic agent comprises vascular endothelial growth factor, fibroblast growth factor, hepatocyte growth factor, stromal derived growth factor, platelet derived growth factor, erythropoietin or combinations thereof. In some embodiments, the therapeutic agent comprises peripheral blood-derived or bone marrow-derived stem cells, mesenchymal stem cells, or marker-specific subsets of bone marrow cells with angiogenic properties. In some embodiments, the therapeutic agent comprises peptides, antibodies, and/or aptamers that recognize specific targets associated with PAD pathology.

In some embodiments, the cellular membrane is derived from a pericyte cell. In some embodiments, the pericyte cellular membrane is engineered to express the ICAM1 binding ligand. In some embodiments, the pericyte cellular membrane expresses the ICAM1 binding ligand.

LFA-1 (Lymphocyte Function-Associated Antigen-1) is a heterodimeric β₂ integrin receptor expressed on the surface of leukocytes. LFA-1 is composed of alpha L (CD11a) and beta (CD18) subunits. ITGAL (Integrin alpha L) is a gene encoding the alpha L subunit (CD11a); the beta subunit is encoded by ITGB2. The general region of the alpha subunit of LFA-1 that binds to ICAM is the I-domain. The LFA-1 complex can bind to ICAM-1, which is expressed on endothelial and antigen-presenting cells. This interaction, protein-protein, specifically a ligand-receptor binding interaction involving integrin-mediated cell adhesion, plays a role in various physiological and pathophysiological processes, particularly in inflammation responses. LFA-1 is known to bind to each of ICAM-1, ICAM-2, ICAM-3, ICAM-4, ICAM-5, and JAM-A. As disclosed herein, the LFA-1/ICAM interaction can be used to target the membrane-coated nanoparticles disclosed herein. Thus, according to the methods disclosed herein, a minimal binding domain or peptide motif that retained ICAM-1 binding capability was identified and the related ITGAL fragment was inserted in a lentiviral plasmid backbone. Consistent with the methods disclosed herein, the cellular membrane was engineered to express a binding fragment of LFA-1. When provided as a therapeutic, the TPM-DNPs comprising a cellular membrane engineered to express a binding fragment of LFA-1, which can target one or more of ICAM-1, ICAM-2, ICAM-3, ICAM-4, ICAM-5, and JAM-A.

Mac-1 (macrophage-1 antigen) is a heterodimeric β₂ integrin wherein one protein subunit is integrin alpha M (CD11b) and the other subunit is CD18. The alpha subunit binds to ICAMs, including ICAM1. Thus, consistent with the methods disclosed herein, the cellular membrane can be engineered to express a binding fragment of CD11b (ITGAM, Integrin alpha M) via incorporation of the ITGAM fragment in a lentiviral plasmid backbone. The engineered cellular membrane, such as a pericyte membrane, can be coated on the nanoparticles as disclosed herein.

Following similar method steps, genes encoding for expression of fibrinogen or hyaluronan can be incorporated in a plasmid, which are incorporated into the membrane of a pericyte cell, yielding a pericyte cell comprising an ICAM1 binding ligand, wherein the ICAM1 binding ligand comprises one or more of an integrin, fibrinogen, hyaluronan, or a binding fragment thereof. In some embodiments, pericyte membrane comprises one or more of LFA-1, Mac 1, or a binding fragment thereof. In some embodiments, the ICAM1 binding ligand binds to one or more of ICAM-1, ICAM-2, ICAM-3, ICAM-4, ICAM-5, and JAM-A.

In some embodiments, the ICAM1 binding ligand comprises an integrin, fibrinogen, or hyaluronan, or a binding fragment of any of the foregoing. In some embodiments, the ICAM1 binding ligand comprises LFA-1 or Mac-1, or a binding fragment of any of the foregoing. In some instances, the ICAM1 binding ligand comprises LFA-1. In some instances, the ICAM1 binding ligand binds to one or more of ICAM-1, ICAM-2, ICAM-3, ICAM-4, ICAM-5, and JAM-A.

In another aspect, the present application discloses a composition comprising: a nanoparticle defining an interior volume and an exterior surface; a therapeutic agent disposed within the interior volume of the nanoparticle; and an engineered pericyte cellular membrane coating disposed on or encapsulating the exterior surface of the nanoparticle. In some embodiments, the engineered pericyte cellular membrane coating comprises an ICAM1 binding ligand. In some instances, the engineered pericyte cellular membrane comprises a binding fragment of LFA-1 can target one or more of ICAM1, ICAM2, ICAM3, ICAM4, ICAM5, and JAM-A; in other instances, it comprises a binding fragment of CD11b. In still other instances the engineered pericyte cellular membrane comprises one or more of an integrin, fibrinogen, hyaluronan, or a binding fragment of any of the foregoing. In some embodiments, engineered pericyte cellular membrane comprises one or more of LFA-1, Mac 1, or a binding fragment of either of the foregoing.

In yet another aspect, the present application discloses a method of manufacturing a membrane-coated nanoparticle, wherein a therapeutic agent is disposed within the interior volume of the nanoparticle; a cellular membrane coating extracted from a pericyte cells is disposed on or encapsulating the exterior surface of the nanoparticle; and the nanoparticle comprises a polymer; the method comprising obtaining a nanoparticle comprising a polymer; loading the nanoparticle with a therapeutic agent; coating a cellular membrane on the nanoparticle, wherein the cellular membrane comprises an ICAM1 binding ligand. In some embodiments, the method further comprises engineering the pericyte cell membrane to express an ICAM1 binding ligand.

In some embodiments, the cell based therapies are incorporated into the nanoparticles disclosed herein and include stem cells, including one or more of peripheral blood-derived or bone marrow-derived stem cells, mesenchymal stem cells, or marker-specific subsets of bone marrow cells with angiogenic properties.

In some embodiments the pro-angiogenic agents incorporated into the membrane coated nanoparticles disclosed herein include small molecule therapeutics, regenerative factors, protein peptides and/or plasmids for gene therapy. In some embodiments, the pro-angiogenic agents include one or more of vascular endothelial growth factor (VEGF), fibroblast growth factor (FGF), hepatocyte growth factor (HGF), stromal derived growth factor (SDF), and platelet derived growth factor (PDGF). Such therapeutics, for example VEGF, FGF, IGF, and PDGF, as well as gene therapy of VEGF, HGF, FGF, HIF-1α, and SDF-1 can be included within the membrane-coated nanoparticles disclosed herein. The incorporation of erythropoietin (EPO) is exemplified herein. EPO, a 34 kDa cytokine produced in adult kidney is expressed by hypoxic ECs and considered an outstanding proangiogenic factor for its association with vessel formation in the heart and brain and for its suppression of pro-apoptotic gene activation in carcinomas.

Without being bound by theory, directing therapeutic agents specifically to the affected tissues and/or cells helps improve therapeutic outcomes in PAD by improving drug localization, enhancing efficacy, and reducing adverse effects. In some embodiments, therapeutic agents included in the engineered membrane-coated nanoparticles disclosed herein include peptides, antibodies, and/or aptamers that recognize specific targets associated with PAD pathology.

In some instances, functional groups such as antibodies and aptamers can be added to the outer surface of the membrane coating the nanoparticles disclosed herein to enhance site targeting, such as to cell surface epitopes found in cancer cells or an anti-ICAM1 antibody.

In some embodiments, the nanoparticle disclosed herein comprises a coating of a cellular membrane derived from a pericyte and an inner core comprising poly(lactic-co-glycolic acid) (PLGA). In some embodiments, the cellular membrane is engineered to express an ICAM1 binding ligand.

In some embodiments, the ICAM1 binding ligand comprises one or more of an integrin, fibrinogen, hyaluronan, or a binding fragment of any of the foregoing. In some embodiments, the ICAM1 binding ligand comprises one or more of LFA-1, Mac 1, or a binding fragment of either of the foregoing.

### Section II. Pharmaceutical Compositions

In one aspect, the present application discloses a pharmaceutical composition comprising an effective amount of any membrane-coated nanoparticle composition disclosed herein, including in Section I, and a pharmaceutically acceptable carrier or excipient. In some embodiments, the nanoparticle is a polymeric nanoparticle. In some embodiments, the cellular membrane is derived from a pericyte cell. In some embodiments, the ICAM1 binding ligand is an integrin, fibrinogen, or hyaluronan, or a binding fragment of any of the foregoing. In some embodiments, the ICAM1 binding ligand comprises LFA-1 or Mac-1, or a binding fragment of either of the foregoing.

In one aspect, provided are compositions comprising an engineered membrane-coated nanoparticle described herein. A composition used in a pharmaceutical composition described herein may be any composition described in Section I. In general, an engineered membrane-coated nanoparticle described herein is mixed with a suitable carrier or excipient in a therapeutically effective amount. By a “therapeutically effective dose”, “therapeutically effective amount”, or, interchangeably, “pharmacologically acceptable dose” or “pharmacologically acceptable amount”, it is meant that a sufficient amount of the membrane-coated nanoparticle and a pharmaceutically acceptable carrier, will be present in order to achieve a desired result, e.g., treating a disease such as PAD (as further described herein).

In general, the engineered membrane-coated nanoparticle containing a therapeutic described herein will be administered in a therapeutically effective amount by any of the accepted modes of administration. The actual amount of the therapeutic contained in the membrane-coated nanoparticle, i.e., the active ingredient, will depend upon numerous factors such as the severity of the disease to be treated, the age and relative health of the subject, the potency of the therapeutic used, the route and form of administration, and other factors. The membrane-coated nanoparticle can be administered according to any suitable dosage regimes, such as once, twice, three times, or four times, etc. a day, or as needed. All of these factors are within the skill of the attending clinician. In some embodiments, the membrane-coated nanoparticle is administered one or more times during a treatment cycle. In further embodiments, the treatment cycle is 21 days. In other embodiments, the treatment cycle is 28 days. In some embodiments, the membrane-coated nanoparticle is administered one or more times during a treatment cycle for up to four treatment cycles.

Therapeutically effective amounts of the therapeutic contained within the engineered membrane-coated nanoparticle may be based on the therapeutic or based on the membrane-coated nanoparticle containing the therapeutic and may range from approximately 0.03 to 50 mg per kilogram body weight of the recipient per day; for example, about 0.1-25 mg/kg/day, or from about 0.5 to 10 mg/kg/day. Thus, for administration to a 70 kg person, the dosage range can be about 1-3,500 mg per day.

In some of the embodiments of the technology described herein, the pharmaceutical compositions are packaged in unit dosage form. The unit dosage form is effective in treating a disease and/or disorder. Generally, a unit dosage including engineered membrane coated nanoparticles disclosed herein will vary depending on patient considerations. Such considerations include, for example, age, protocol, condition, sex, extent of disease, contraindications, concomitant therapies, and the like. An exemplary unit dosage based on these considerations can also be adjusted or modified by a physician skilled in the art. For example, a unit dosage for a patient comprising the present technology can vary from 3×10⁻⁵ g/kg to 1 g/kg, preferably, 1×10-3 g/kg to 1.0 g/kg. Dosage can also vary from 0.01 mg/kg to 100 mg/kg or, preferably, from 0.1 mg/kg to 10 mg/kg. In some embodiments, the unit dosage comprises 0.01 mg/kg to 0.5 g/kg. In some embodiments, the unit dosage comprises 0.01 mg/kg to 100 mg/kg. In some embodiments, the unit dosage comprises 0.01 mg/kg to 50 mg/kg. In some embodiments, the unit dosage comprises 0.01 mg/kg to 10 mg/kg. In some embodiments, the unit dosage comprises 0.01 mg/kg to 5 mg/kg. In some embodiments, the unit dosage comprises 0.1 mg/kg to 0.5 g/kg. In some embodiments, the unit dosage comprises 0.1 mg/kg to 100 mg/kg. In some embodiments, the unit dosage comprises 0.1 mg/kg to 50 mg/kg. In some embodiments, the unit dosage comprises 0.01 mg/kg to 10 mg/kg. In some embodiments, the unit dosage comprises 0.1 mg/kg to 5 mg/kg.

In general, the nanoparticle formulations described herein will be administered as pharmaceutical compositions by any one of the following routes: oral, transdermal, intranasal, by suppository, parenteral (e.g., intramuscular, intravenous or subcutaneous), inhalation, or intrathecal administration. Compositions can take the form of tablets, pills, capsules, semisolids, powders, sustained release formulations, solutions, suspensions, elixirs, aerosols, or any other appropriate compositions. For delivery via inhalation, the composition can be formulated as liquid solutions, suspensions, aerosol propellants, or dry powder and loaded into a suitable dispenser for administration. The choice of formulation depends on various factors such as the mode of drug administration and bioavailability of the drug substance.

In some embodiments, pharmaceutical compositions described herein are comprised of, in general, the nanoparticle formulation described herein in combination with at least one pharmaceutically acceptable excipient. In some instances, acceptable excipients are non-toxic, aid administration, and do not adversely affect the therapeutic benefit. Such excipient may be any solid, liquid, semi-solid or, in the case of an aerosol composition, gaseous excipient that is generally available to one of skill in the art.

Solid pharmaceutical excipients include but are not limited to starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, phosphate buffered saline, citrate buffer, aqueous dextrose, glycols, etc.

The amount of a nanoparticle formulation described herein in a pharmaceutical composition can vary within the full range employed by those skilled in the art. In some embodiments, the formulation will contain from about 0.01-99.99 wt % of the nanoparticle formulation based on the total weight of the composition, with the balance being one or more suitable pharmaceutical excipients. Preferably, in some instances, the nanoparticle formulation described herein is present at a level of about 1-80 wt %.

A pharmaceutical composition described herein may be made in any manner not inconsistent with the technical objectives of the current disclosure. For example, in some embodiments, a membrane-coated nanoparticle may be mixed (such as with a high shear mixer) with one or more additional components (such as a pharmaceutically acceptable excipient) to form a pharmaceutical composition described herein.

### Section III. Method of Treatment

In any of the embodiments of the methods described herein, the method may involve the administration of a pharmaceutical composition, where the pharmaceutical composition includes any one of the embodiments of the compositions of the present technology or a pharmaceutically acceptable salt thereof as well as a pharmaceutically acceptable carrier or excipient.

In another aspect the present application discloses a method of treating a condition of a patient in need thereof, the method comprising: disposing any engineered membrane-coated nanoparticle composition disclosed herein within a biological compartment of the patient. In some embodiments, the condition is peripheral artery disease. In some embodiments, the nanoparticle is a polymeric nanoparticle. In some embodiments, the cellular membrane coating the nanoparticle is derived from a pericyte. In some embodiments, the therapeutic agent is erythropoietin. In some embodiments, the nanoparticle releases EPO upon membrane and polymer degradation. In some embodiments, the degradation is affected by hydrolysis, enzymes, pH, etc. In some embodiments, the ICAM1 binding ligand is an integrin, fibrinogen, or hyaluronan, or a binding fragment of any of the foregoing. In some embodiments, the ICAM1 binding ligand comprises LFA-1 or Mac-1, or a binding fragment of either of the foregoing.

In any of the embodiments of the methods described herein, the method may involve the administration of a pharmaceutical composition, where the pharmaceutical composition includes an effective amount of any one of the embodiments of the membrane-coated nanoparticle of the present technology and a pharmaceutically acceptable carrier or excipient. In some embodiments, the effective amount is from about 0.01 μg to about 900 mg of the nanoparticle formulation per gram or milliliter of the pharmaceutical composition. In some embodiments, the effective amount is from about 0.01 μg to about 800 mg of the membrane-coated nanoparticle per gram or milliliter of the pharmaceutical composition. In some instances, the effective amount is from about 0.01 μg to about 700 mg of the membrane-coated nanoparticle per gram or milliliter of the pharmaceutical composition. In some cases, the effective amount is from about 0.01 μg to about 600 mg of the membrane-coated nanoparticle per gram or milliliter of the pharmaceutical composition. In some embodiments, the effective amount is from about 0.01 μg to about 500 mg of the membrane-coated nanoparticle per gram or milliliter of the pharmaceutical composition. In some embodiments, the effective amount is from about 0.01 μg to about 400 mg of the membrane-coated nanoparticle per gram or milliliter of the pharmaceutical composition. In some implementations, the effective amount is from about 0.01 μg to about 300 mg of the membrane-coated nanoparticle per gram or milliliter of the pharmaceutical composition. In some embodiments, the effective amount is from about 0.01 μg to about 200 mg of the membrane-coated nanoparticle per gram or milliliter of the pharmaceutical composition. In some cases, the effective amount is from about 0.01 μg to about 100 mg of the membrane-coated nanoparticle per gram or milliliter of the pharmaceutical composition. In some embodiments, the effective amount is from about 0.1 μg to about 500 μg of the membrane-coated nanoparticle per gram or milliliter of the pharmaceutical composition.

In some embodiments, the effective amount of the therapeutic or composition is 0.5, 1, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 mg or more, including increments therein; for example in some embodiments, the effective amount of the therapeutic or composition is from 10 mg to 500 mg or 25 to 600 mg. In some embodiments, the composition per unit dosage contain from about 0.1% to about 99% of the membrane-coated nanoparticle or the therapeutic itself. In some embodiments, the composition per unit dosage contain from about 10% to about 60% of the membrane-coated nanoparticle or the therapeutic itself.

In some aspects, provided is a kit comprising, consisting essentially of, or consisting of an effective amount of a membrane-coated nanoparticle described herein, and optionally instructions for use. In some aspects, the instructions comprise, consist essentially of, or consist of a description of a method of treatment as described herein.

### EXAMPLES

Cell transfection is the process of introducing foreign nucleic acids (DNA or RNA) into cells. There are variety of methods familiar to those of skill in the art. Non-viral based cell transfection includes (a) chemical-based transfection, exemplified by liposomal-based and non-liposomal-based, and includes using lipid molecules to form lipid-nucleic acid complexes, which can be taken up by cells; (b) physical or mechanical transfection methods, such as electroporation, sonoporation, and gene microinjection. Viral based cell transfection uses engineered viruses used to deliver nucleic acids into cells. Common viral vectors include adenoviruses, lentiviruses, retroviruses, and adeno-associated viruses; stable transduction involves integrating the viral vector's genetic material into the host cell's genome, ensuring long-term gene expression-allowing the transgene to be replicated and passed onto daughter cells during cell division. Retroviruses and lentiviruses are examples of vectors that facilitate stable transduction, as they integrate their genetic material into the host DNA. Transient transduction delivers genetic material into host cells without integrating it into the host genome. This results in temporary gene expression that lasts only until the viral vector is degraded or diluted through cell division.

Lentiviral transduction offers long-term and stable gene expression because lentiviral vectors integrate their genetic material into the host cell's genome, making them effective for applications requiring sustained gene activity. The method boasts high transduction efficiency and can effectively target a wide range of cell types, including non-dividing cells. Additionally, lentiviral transduction simplifies the workflow by reducing the need for large-scale DNA preparations and complex transfection procedures, thanks to the virus's ability to use the host cell machinery for replication and packaging. Lentiviral vectors also have a large payload capacity, allowing for the delivery of larger or multiple genes compared to some other vectors. They are generally safer due to their lower immunogenicity, which minimizes the risk of eliciting an immune response that could interfere with the transgene's function. Moreover, lentiviral transduction causes minimal cytotoxicity, leading to less cellular damage compared to physical methods like electroporation or chemical methods that may be harsher on cells.

Without being bound by theory, the membrane of engineered stem cells made to express targeting moieties reduce risk associated with lentiviral transduction, since the engineered cells are not used in a clinical application, but the biomimetic cell membrane capable of escaping the immune response is used, thereby increasing the targeting capability, which helps overcoming any decreased bioavailability of therapeutic agent. The combination of engineered cell membrane with the nanoparticles disclosed herein provide for the targeted drug delivery system of therapeutic agents. The disclosed systems can further provide a patient specific model when stem cells are obtained from the patient.

As disclosed herein, stem cell-like pericyte-membrane expressing an ICAM1 binding ligand coated onto nanoparticles loaded with EPO were prepared. These systems enhance targeting and increase circulation time, thereby facilitating angiogenesis.

### Example 1

Immortalized mouse pericytes purchased from Applied Biological Materials (ABM Inc.) were cultured in Collagen Type I (Corning) coated cell culture flasks with Prigrow III media, which was obtained from ABM Inc. and contained 10% FBS (Sigma Aldrich, St. Louis, MO) and 1% Pen strep (Life Technologies, Thermo Fisher Scientific, Waltham, MA) at 37° C. and 5% CO₂. In this project, endothelial cells (ECs) were mainly used to test the in vitro therapeutic effects of nanoparticles, including proliferation and tube formation. The lentiviral packaging cell line Lenti-X 293 T was provided by Takara-Clontech (Takara Bio USA, Inc., San Jose, CA) and was cultured in Dulbecco's Modified Eagle's Medium (DMEM) containing 10% FBS (Sigma Aldrich) and 1% Pen strep (Life Technologies) at 37° C. and 5% CO₂.

### Materials

All chemicals, if not specified, were purchased from Sigma-Aldrich. Poly (lactic-co-glycolic acid) (PLGA), LG 50:50, (Mn 25,000-35,000 Da) was obtained from PolySciTech (division of Akina, Inc., Purdue Research Parl, IN) Rhodamine B dye was received from Sigma-Aldrich, and Heptamethine cyanine (Cy7), Cyanine5 (Cy5) and IR 820 were bought from Lumiprobe (Cockeysville, MD). All reagents were of analytical grade.

### Preparation of Polymer Nanoparticles

Drug-loaded PLGA NPs were synthesized using a modified version of standard double emulsion technique as previously described in Xu, Hao, et al., Journal of cardiovascular translational research 5 (2012): 519-527.

Briefly, ˜20000 IU/mL of Erythropoietin was added dropwise to the mixture of 100 mg of PLGA dissolved in 3 ml of Chloroform (oil phase). The solution was dispersed via sonication for 30 seconds at 25 W. After the polymer and drug/protein solution were completely dispersed, the emulsion solution was added dropwise to 25 ml of 5% Polyvinyl alcohol (PVA) solution (water phase) under constant stirring and sonicated for 1 minute at 25 W for emulsification. The organic solvent was evaporated overnight under stirring at room temperature. The particles were collected by centrifugation at 22,00 RPM for 25 mins at 4° C. The supernatant was collected and used to measure protein loading efficiency via an indirect method with the help of a BCA assay kit from ThermoFisher and UV-VIS spectrophotometer (Tecan, Infinite M200, California). The pellet was washed twice and resuspended in 3 mL DI water. The particles were freeze-dried for 24 hours and lyophilized (Labconco, Kansas City, MO) for later use.

Blank nanoparticles were prepared by the same process mentioned above without adding the drug/protein content during the nanoparticle formulation process. For dye-loaded NPs, 10 mg of either Rhodamine or Cy7 dyes were added in the PLGA polymer solution dropwise and the above stated procedure was followed, as further described in Zhukova, Vasilisa, et al., Pharmaceutics 13.8 (2021): 1145.

### Plasmid Purification

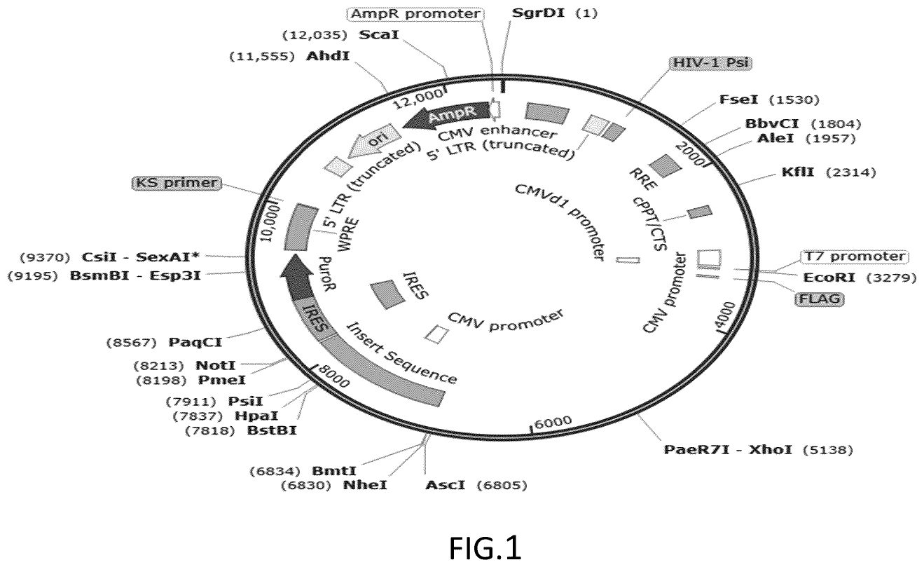

A minimal binding domain or peptide motif from ITGAL that retains ICAM1 binding capability was identified and inserted in the lentiviral plasmid backbone (Actual LFA part of the gene is 3533 base pairs.) Escherichia coli (E. coli) bacteria were transformed with CMV/T7-(K)-eGFP_(K)—SP-FLAGmITGAL_pGenlenti plasmid (Genscript Biotech, Piscataway, NJ) as shown in FIG. 1, and packaging plasmids such as pMDLg/pRRE, pRSV-Rev and pMD2.G (Addgene, Watertown, Massachusetts). As shown, the insert sequence in the plasmid refers to the ICAM1 binding ligand. The plasmid has flag tag and GFP protein for tracking transfections and ICAM1 binding ligand transmembrane protein to enhance angiogenesis. The plasmid backbone also contains an Ampicillin gene, which helps bacterial culture selection.

E. coli bacteria were grown in the standard Luria Bertani (LB) medium with ampicillin antibiotic (100 mg/l) shaken 180 RPM at 37° C. for 14-16 hours. Bacteria pellets were harvested by centrifuging at 6000 g for 15 minutes at 4° C.; then plasmids were purified by using EndoFree Plasmid Giga (Qiagen) kits following the manufacturer's instructions. The purified plasmids were redissolved in Tris-EDTA (TE) buffer and was stored in −80° C. The entire plasmid purification process was described in Qiagen plasmid purification kit protocols.

### Plasmid Characterization

Plasmid production and purification were measured via nanodrop (Thermo Scientific 2000 1-position Spectrophotometers). About one drop (2 μl) of samples was used for measuring absorbance. The blank TE buffers were used to measure the background before reading plasmid samples for accurate measurements. The software built in the device analyzed the absorbed data to provide the plasmid concentration and ratios of 260 nm/280 nm for assessing purity of plasmids from protein and phenol contamination and 260 nm/230 nm for assessing purity of plasmids from high salt contamination. Plasmid structural integrity was determined by running Agarose Gel Electrophoresis. The digested and undigested plasmids were mixed with 6×DNA loading buffer to electrophorese at 110 V for 30 minutes and imaged using the BIO-RAD ChemiDoc Touch Imaging System. The digested samples were cut at two sites using NheI and EcoRI enzymes to separate the ICAM1 binding ligand genes from the Lentiviral backbone. 1 kb plus DNA ladder (BioLabs, Cambridge, Massachusetts) was used for this gel electrophoresis analysis.

### Lentiviral Vector Production, Transduction and Sorting of Engineered Pericytes

CMV/T7-(K)-eGFP_(K)—SP-FLAGmITGAL_pGenlenti lentiviral plasmid (Genscript) (used as backbone for expressing ICAM1 binding ligand) and packaging plasmids were used. The lentivirus was produced via transection of LentiX 293T cells with CMV/T7-(K)-eGFP_(K)—SP-FLAGmITGAL_pGenlenti expression vectors and pMDLg/pRRE, pRSV-Rev and pMD2.G (3rd Generation) the viral packaging plasmids using Lipofectamine 3000 (Invitrogen, Thermo Fisher Scientific). Supernatants containing lentivirus were collected 24, 48, and 72 hours later. The collected virus was centrifuged, and the supernatant was saved.

### Lentiviral Transduction of Pericytes

For transduction of pericyte cells, cells were thawed, and culture was started for lentiviral transduction. At day 1, pericytes were co-cultured for 24 hours with lentiviral supernatants from Day 1 in the presence of 8 μg polybrene (Merck, Rahway, NJ). On 2nd day, the media was removed and the lentiviral particles from Day 2 were added into a 6 well plate, and the process was repeated for Day 3. After transduction, the cells were cultured in the presence of 10% FBS until use. Pericytes surface markers were later detected via anti-CD146 antibodies (Biolegend, San Diego, CA) using flow cytometry methods. Pericytes expressing ICAM1 binding ligand on the cell surface were sorted 2 times via a sorter at Baylor School of Medicine (Fluorescence Activated Cell Sorting (FACS) Aria Cell sorter) with the help of GFP expression of plasmids. After expansion of sorted pericytes, the cell line was expanded and was sorted for the 3rd time using anti-Flag tagged antibody (Biolegend, FITC fluorescence). ICAM1 binding expression was detected by primary antibody mouse biotinylated ICAM1 protein (Sino Biological, Beijing, Chin), and secondary antibody streptavidin tagged to fluorescence (Biolegend, APC-tagged) to confirm the presence of engineered cells expressing ICAM1 binding ligand using flow cytometry.

### ICAM1 Binding Ligand Membrane Isolation

Cells (about 40 million) (Transfected and non-transfected cells) were washed three times with cold PBS (1×). Before the last spin, cells were counted and resuspended in cold hypotonic buffer Tris-HCl (10 mM, pH7.5) in 40 mL. Protease and phosphatase inhibitor cocktail (PIC) was added (5 μL/million cells) and incubated with samples at 4° C. for 20 minutes. The mixture was centrifuged at 4000 g for 10 minutes. The resulting pellet was resuspended in cold PBS 0.25X+PIC at 1 ml/10 million cells and incubated for another 20 minutes at 4° C. The mixture was centrifuged at 4000 g for 10 minutes, then the pellet was resuspended in cold PBS 1X+PIC and kept on ice. DNAse reaction was performed on the samples based on their DNA contents (for 20 million cells: 20 IU DNAse for 4 hours). After using DNAse, mixture was centrifuged at 4000 g for 10 minutes. The isolated membranes were collected, lyophilized overnight and stored at −20° C. Right before use, the membrane was weighed and resuspended in a PBS solution.

Coating NPs with Pericyte Membranes Expressing ICAM1 Binding Ligand and Nanoparticle Characterization

ICAM1 binding ligand membrane-coated NPs were prepared as follows; drug-loaded PLGA nanoparticles were added via hydrating these NPs with freeze-dried membranes expressing ICAM1 binding ligand (1:1, w/w ratio). Generally, the nanoparticles were core loaded with EPO protein. The core nanoparticles were then coated with membranes isolated from engineered pericytes. This coating process was achieved through extrusion using a syringe and filter resulting in the formation of membrane-coated nanoparticles. The mixture was sonicated for 1 minute using a probe sonicator (QSonica, Connecticut). Subsequently, the mixture was co-extruded using an Avanti mini extruder (Avanti Polar Lipids, Alabaster, AL), as generally described in Yaman, S., et al., Bioactive Materials 2024, 34, 422-435. The confirmation of coating of membrane onto nanoparticles was done by using membrane dye DIL (ThermoFisher, lipophilic membrane stain) and flow cytometry (BD Biosciences, Franklin Lakes, NJ).

### Physiochemical Properties of TPM-DNPs

The size distribution, zeta potential and polydispersity of the NPs were measured using NanoBrook 90Plus PALS analyzer (Brookhaven Instruments, Holtsville, NY) and Zeta view (Particle Metrix, Mebane, NC) for measuring number of particles. For DLS measurements, 50 μL of 1 mg/mL Nanoparticle (“NP”) suspension was added to a transparent cuvette containing 3 mL of DI water, and the size measurement was performed using a laser. Zeta potential (charge) measurements were done using a probe attachment placed inside the cuvette.

The surface morphology of the formulated nanoparticles was visualized by transmission electron microscopy (TEM). Briefly, freeze-dried nanoparticle samples in DI water were fixed onto ozone treated copper grids and stained with uranyl acetate (0.5%). A H-7500 TEM transmission electron microscope (Hitachi, Santa Clara, California) was used to visualize the particle morphologies.

### Drug Loading of TPM-DNPs

To study the loading efficiency, the amount of protein in the supernatant during TPM-DNP fabrication was measured by BCA assays (ThermoFisher) following the manufacturer's instructions. The efficiency was determined as the loaded amount against the total amount used for the fabrication (Equation 1).

$$Drug loading efficiency ( \% ) = \frac{Initial drug used - drug in supernatant}{Initial drug used} * 100 \qquad ( 1 )$$

### Drug Release Kinetic of TPM-DNPs

To study the drug release kinetics of TPM-DNPs, 4 mg of TDNPs were dispersed in 4 ml of PBS with a pH of 7.4. The resulting solution was divided equally into 4 tubes, and NPs was suspended in PBS solution inside a dialysis membrane with a molecular weight cut-off (MWCO) larger than that of the MW of the drug. The released protein contents in samples, which were collected at predetermined time points (1 hour, 6 hours, 12 hours, 24 hours, 3 days, 5 days, and 10 days), were analyzed using BCA assay kits (ThermoFisher).

### Stability of TPM-DNPs

The stability of TPM-DNPs was determined in reference to size changes in various fluids including PBS, media containing serum, 0.9% NaCl (saline), and human simulated body fluid (SBF) over a period (0, 1, 12, and 24 hours for a short time as well as 3, 5, 7, and 10 days for a long time) to reveal any particle aggregation as previously described in Yaman, Serkan, et al., Frontiers in bioengineering and biotechnology 8 (2020): 943. At each designated time-point, the sizes of NPs were measured using NanoBrook 90Plus PALS analyzer (Brookhaven Instruments, Holtsville, NY) and was compared with blank NPs.

### Confirmation of Ligands on the Extruded NPs Through Binding Affinity Study

The binding characteristics of TPM-DNPs were studied using ResoSens label-free optical detection system. In this study, TPM-DNPs, PM-DNPs and anti-ICAM1 (Biolegend) antibody-coated NPs were used for the ICAM1 binding analysis. Blank NPs were used as the subject (control) groups, and the binding affinity towards the Biotinylated ICAM1 protein-coated surface (ACRO Biosystems, Newark, DE) were measured in terms of resonance shift as described in Lin, Arthur, et al., Journal of Biomedical Materials Research Part A: An Official Journal of The Society for Biomaterials 93.3 (2010): 833-842.

### Results

Current pro-angiogenic strategies, including delivery of growth factors and growth factor-encoding genes, have proven beneficial for treating PAD in preclinical studies; however, they failed to improve primary outcomes in Phase 2 and 3 clinical trials including the delivery of growth factors: VEGF, FGF, IGF, and PDGF, and VEGF, HGF, FGF, HIF-1α, and SDF-1 gene therapy. These treatments have some limitations including low therapeutic effects and limited protein availability over time, mostly due to a short half-life and enzymatic degradation, off-target deposition (with systemic delivery), and ineffectiveness in ischemic tissues. To overcome these challenges, ICAM1-binding ligand expressed pericyte membrane-coated, EPO-loaded NPs that target injured tissues, mimic the interactions between pericytes and endothelial cells, mediate capture of EPCs to provide targeted and controlled release of therapeutic protein EPO to these cells, and facilitate tissue regeneration for treating PAD effectively were developed.

Pericyte membranes expressing ICAM1 binding ligand was selected for coating onto EPO-loaded NPs in part because ICAM1 is highly expressed on endothelial cells during the ischemic conditions. The hybrid biofunctionalized and biomimetic stem cell like membrane-coated NP system serves as a universal drug carrier that can load any drug and can be used as a nanocarrier system for different diseases, including cardiovascular diseases.

### Example 2

### Fabrication, Purification and Characterization of ICAM1 Binding Ligand

### Plasmid Purification and Characterization

Four different plasmids such as CMV/T7-(K)-eGFP_(K)—SP-FLAGMITGAL_pGenlenti, and packaging plasmids, including pMDLg/pRRE, pRSV-Rev and pMD2.G, were successfully purified by EndoFree Qiagen kits as described in Liu, Chengyu, et al., Lipoproteins and Cardiovascular Disease: Methods and Protocols (2013): 203-215. Then 1.5 mg CMV/T7-(K)-eGFP_(K)-SP-FLAGmITGAL_pGenlenti, 6 mg pMDLg/pRRE, 6 mg pRSV-Rev, and 6 mg pMD2.G per kit. The purification of lentiviral plasmid is described as the purity of the collected plasmids in Table 1. The absorption of light at 260/280 nm ratio relates to protein contamination in the samples while 260/230 nm ratio relates to salts or other compounds in the samples. The results showed that the purified plasmids were in the acceptable range and are good to load inside NPs.

TABLE 1
Plasmid characterization

| Parameter | pGenlenti | Normal Acceptable Range |
|---|---|---|
| 260 nm/280 nm ratio | 1.9 | ~2.0 |
| 260 nm/230 nm ratio | 2.0 | 2.0 to 2.22 |

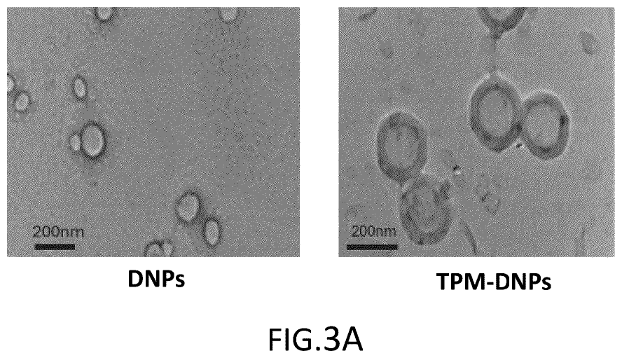

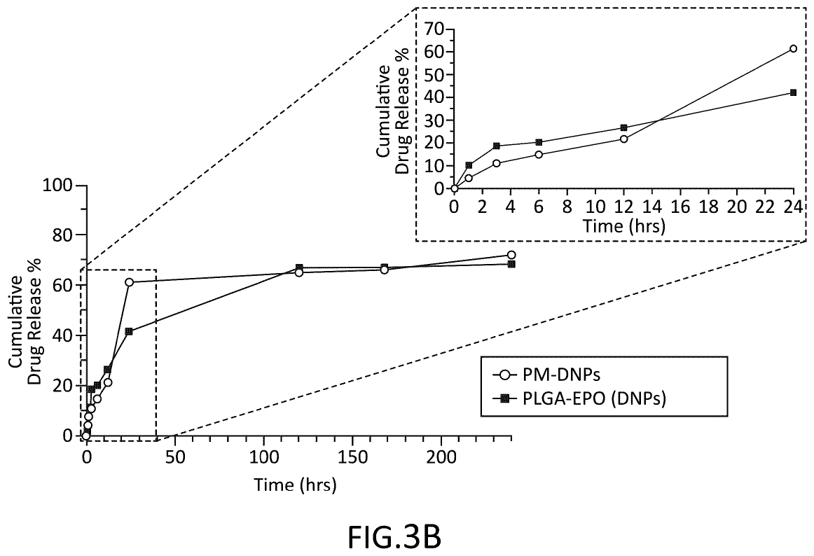

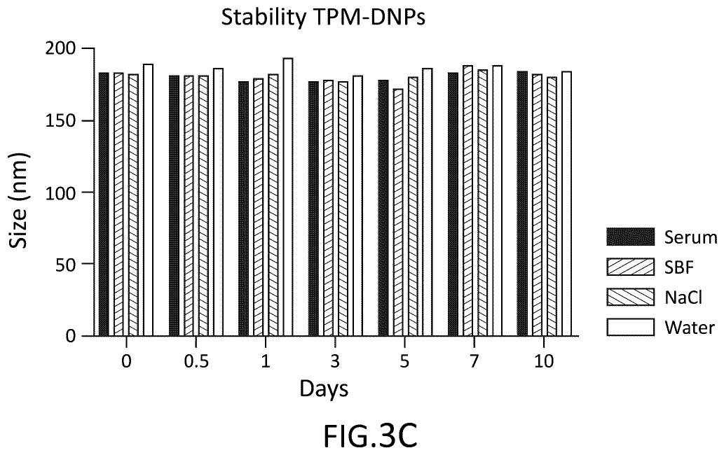

Gel electrophoresis (FIG. 3) showed wide bands of the supercoil (undigested) pGenlenti plasmids. The digested pGenlenti plasmids separated two bands on the gel including the backbone (larger) band at the same level and the ICAM1 binding ligand gene (shorter) band sized close to 2.5k base pairs, which is the actual size of ICAM1 binding ligand insert.

### Lentiviral Vector Production

Lipid-based plasmid DNA transfection methods were followed for lentiviral transfection studies. Lenti-X cells (500,000 cells/well) were seeded on 6-well-plates overnight in 10% FBS DMEM. Purified plasmids (5 μg) with lipofectamine 3000 and packaging plasmids were added and incubated for 2 hours in 1% FBS in DMEM for gene transfection studies. Then Lenti-X cells were supplemented with 10% FBS DMEM. GFP expression protein was observed under fluorescent imaging system over time. The transfection was confirmed through the expression of GFP on transfected cell line in microscope. After transfection, constitutive GFP fluorescence was observed, and culture supernatant was collected over the course of 3 days. Cell culture supernatants were then used to transduce immortalized mouse pericyte cells to express Flag tag, GFP and anti ICAM1 proteins.

### Lentiviral Transduction of Mouse Pericytes

The collected viral particles from the transfected Lenti-X were added to the mouse immortalized pericytes with polybrene for 48 hours. After viral transduction, pericytes showed the transfection expression via fluorescent expression of GFP included in the plasmid backbone. About 60% of the pericytes were transfected and expressed GFP and were also sorted using Live/Dead stain kit-Fixable viability Dye eFluor (V450, Invitrogen). 60% of the population were sorted based on the GFP expression, and the live cells were grown and expanded. These populations were confirmed and characterized via flow cytometry based on the expression of peptide protein (flag protein) that is a part of ICAM1 binding ligand insert sequence as described previously by Stoner, Samuel A., et al., Cytometry Part A 89.2 (2016): 196-206. About 27% of the cells expressed flag protein from the 60% GFP positive population. The expanded cells were sorted using anti-flag tag antibody tagged to APC fluorescence. These cells expressed GFP and flag tag indicated that the sorted population has both GFP and flag tag.

### ICAM1-Binding Receptor Confirmation in Pericyte Cell Membranes

After confirmation of reporter plasmid transduction and stable GFP and flag protein expression, the same set of experiments was used for the ICAM1 binding ligand that is specific to ICAM1 receptor expressed by ECs in inflammatory conditions. In this case, APC tagged secondary (2°) antibody was used to tag to primary biotinylated mouse ICAM1 protein to detect the ICAM1 binding ligand through flow cytometry. Non-specific mouse antigen protein tagged to 2° antibodies tagged to APC was used as control group to determine any non-specific binding of the ligand molecule.

Pericytes cells were successfully transduced to express anti ICAM1 ligand on their cell surface. A distinctive shift was seen in the anti-biotinylated-APC channel compared to the non-transduced anti-biotinylated secondary antibody. The control groups included non-specific mouse antigen and secondary antibody only group. Based on the flow cytometry results, ICAM1 binding ligand transduced cells showed a distinctive shift compared to the non-transduced pericyte cell group and having more than 95% positive cell population. As transduction efficacy, over 90% of all the cell population were found to express ICAM1 binding antigen on the surface.

Confirmation of Active Membrane Receptors on Pericytes after Cell Transduction, Sorting and Expanding

After pericyte cells were transduced with ICAM1 binding ligand, sorted and expanded, the cell line was checked for the integrity of the cell surface receptor to determine if the transduction process had caused any changes in the physiology state of the cell line using flow cytometry as generally described in Pickles, S., et al., J Vis Exp 91:51887, 2014. Anti-CD146 tagged to FITC fluorescent was used to bind to the CD-146 receptor expressed by pericytes. The results indicate that 97.8% of the cells retained the physiological state of the cells after transduction and sorting when compared to unstained cells.

### Physical Characterization of TPM-DNPs

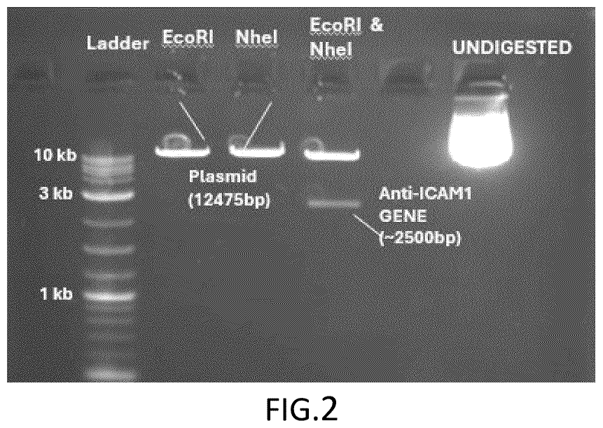

Engineered membranes expressing ICAM1 binding ligands, EPO-loaded NPs (TPM-DNPs) were fabricated and characterized and the membrane expressing ICAM1 binding ligands was extruded with NPs to formulate membrane coated NPs. After coating the pericyte cell membrane onto the PLGA NPs, the physio-chemical characteristics of TPM-DNPs were analyzed (FIG. 2) using methods previously described in Pandey, Nikhil, et al., Advanced healthcare materials 7.7 (2018): 1701069. TPM-DNPs were formulated with the loading of EPO protein. Dynamic light scattering (DLS) analysis demonstrated that the membrane coated NPs had a particle size ranging from 200-250 nm, generally around 224 nm, and they were larger compared to that of the DNPs (˜200 nm), probably due to the membrane coating. The membrane coating also exhibited a zeta potential showing more negative value than bare nanoparticles. Though there was a particle size difference after coating of the cell membranes, there was no significant change in the polydispersity with all the groups having a good polydispersity index (PDI) well below 0.3 (Table 2).

TABLE 2
Nanoparticle Properties

| Nanoparticle | Size | PDI | Zeta potential | Loading Efficiency |
|---|---|---|---|---|
| DNPs | 201 ± 32 | 0.130 | −14.0 ± 15 | 82 |
| TPM-DNPs | 224 ± 38 | 0.205 | −25.2 ± 21 | 77 |

These NPs consisted of spherical structures (FIG. 3A) with a clear visualization of the membrane coating seen by the transmission electron microscopy (TEM) analysis. The method disclosed herein effectively encapsulated protein inside the NP core (≥80% loading efficiency). The coating efficiency of the PM-DNPs was confirmed by flow cytometry using DiD membrane dye (Red), showing about 65% of the NPs were coated with cell membranes. In addition, these NPs provided a stable sustained EPO release with almost 62% of drug released in up to 10 days (FIG. 3B). The drug release kinetics displayed an initial burst release followed by a sustained release in up to 10 days of incubation at 37° C. in PBS. The rate of release kinetics differed across DNPs and TPM-DNPs. Without being bound by theory, the TPM-DNPs might have had a delayed release due to the presence of membrane coated onto NPs that slowed down the degradation process of the PLGA NPs. The particles were stable at time points 0, 0.5, 1, 3, 5, 7, and 10 days when constituted in different reagents such as simulated body fluids, serum, water, and 0.9% NaCl (FIG. 3C), thereby ensuring minimal particle aggregation potential when encountered in the human body.

### ICAM1 Binding Ligand Expression Confirmation on Extruded NPs

For the confirmation of ICAM1 binding ligand of extruded NPs, TPM-DNPs were checked on ResoSens label-free optical detection instrument as described in Yaman, Serkan, et al., Frontiers in bioengineering and biotechnology 8 (2020): 943.

The sensor plates in a 96-well microarray format have the sensor element embedded in the bottom of the plate. The reaction occurs at the sensor surface and is compatible with conventional attachment chemistries such as biotinylated reagents. The reflected beam is monitored from the bottom of the plate, without having to penetrate through the sample. The biotinylated ICAM1 protein binds to the neutravidin coated microarray plate and the affinity of the test group towards the protein is measured. The TPM-DNPs had significantly higher affinity than the other groups because of the presence of ICAM1 binding ligands on these NP surfaces.

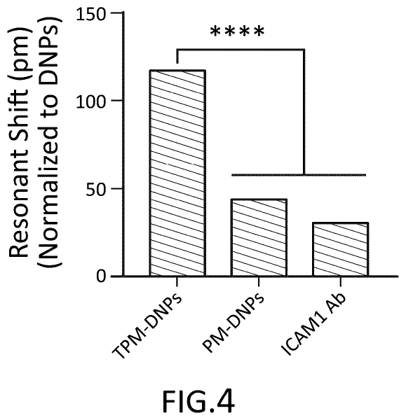

As shown in the graph of binding affinity (see FIG. 4), TPM-DNPs bound onto ICAM1 protein coated onto the surface of immobilized neutravidin 96-well plates. TPM-DNPs showed higher binding strength when compared to the PM-DNPs and anti-ICAM1 antibody at 1:1 (w/w NP: antibody), and there was a shift of about 70 ppm when compared to PM-DNPs. These binding kinetic results not only confirmed the higher presence of the specific ICAM1 binding ligand on the nanoparticle coating, but also its ability to bind to its specific target i.e. ICAM1 receptor.

### Discussion

As shown herein, the pericyte cell line expressing ICAM1 binding ligand was prepared via lentiviral transduction. In transduction studies, the Lenti-X 293T cell line was used to produce lentiviral particles carrying the ICAM1 binding ligand gene. These particles were transduced into pericyte cells. The presence of the ICAM1 binding ligand and CD-146 cell surface marker were confirmed through flow cytometry.

Pericyte cells expressing ICAM1 binding ligand were fabricated and characterized, and then the cell membrane were isolated and extruded with PLGA NPs to formulate TPM-DNPs. The zeta potential of the TPM-DNPs was approximately −25.2 mV, which was more negative than DNPs (−14.0 mV), and indicates that the presence of membranes coated over the NPs imparts more negativity to the particles. The size of the TPM-DNPs was monitored using dynamic light scattering and was found to be 224 nm, with approximately a 23-nm increase compared to the DNPs (201 nm).

The loading efficiency of the TPM-DNPs was observed to be approximately 77% and the drug release kinetics displayed a biphasic release with an initial burst release followed by a sustained release up to 10 days of incubation at 37° C. in PBS (pH 7.2). The rate of release kinetics differed across DNPs and the TPM-DNPs. Without being bound by theory, this might be due to the diffusion barrier effect of the membrane layer. In addition, TEM images revealed that membrane coated NPs had shown as a core shell, which confirms the membrane coating on the surface of DNPs. The coating efficiency of TPM-DNPs compared to unstained control, stained TPM-DNPs samples displayed a significant fluorescence intensity shift (˜60%) using flow cytometry. Without being bound by theory, this could be due to the loss of membrane and NPs during the extrusion process. To identify the specific ICAM1 binding ligand in the TPM-DNPs formulations, the NPs were analyzed using ICAM1 protein through a binding affinity study and the binding affinity of TPM-DNPs were 4 times higher than that of anti-ICAM1 antibodies.

### Example 3

### Evaluation of Engineered Pericyte Cell Membrane Coated Nanoparticles In Vitro.

Membranes isolated from engineered pericyte cells coated on PLGA nanoparticles to form TPM-DNPs were evaluated in terms of cell viability assay, cytocompatibility, cell migratory properties and facilitation of angiogenesis. The cell targeting ability of TPM-DNPs were evaluated using activated mice endothelial cells (ECs) by adding tumor necrosis factor-alpha (TNP-α) to mimic inflamed endothelial cells by overexpressing ICAM1 receptor in vivo using cell uptake studies and microscopic analysis. These studies proved the membrane coating on NPs and ligand existence on their surface. The in vitro killing efficacy of TPM-DNPs was assessed against ICAM1 overexpressing cell lines in vitro and compared with other treatment groups. Endothelial cells migrate into the surrounding tissue in response to angiogenic chemokines. The ECs migratory properties towards the injured endothelium was assessed by creating injury in a 2D matrix and the migration of ECs was assessed by cell movement towards the endothelial cells in an attempt to close the wound created. The tube formation stage of angiogenesis can be modeled in vitro by plating endothelial cells with extracellular matrix components. The morphogenesis of ECs were quantified by measuring the formation of length or area of capillary-like structures as described in Le, Duong Q., et al., Scientific reports 7.1 (2017): 8692.

### Material and Method

### Cell Lines and Culture Conditions

Mouse primary endothelial cells (ECs) were obtained from American Type Culture Collection (ATCC) and cultured in Dulbecco's Modified Eagle's Medium (DMEM) containing 10% FBS (Sigma Aldrich) and 1% Pen strep (Life technologies) at 37° C. and 5% CO₂. EC culture media were renewed every two days.

### Cell Uptake Studies Under Static Conditions

For fluorescence uptake microscopy imaging studies using Imaging system (Echo microscope), mouse endothelial cells (ECs) were cultured on glass-bottomed six-well tissue culture plates (Corning). After 24 hours, the cells activated using Tumor necrosis factor-α (TNF-α) at a concentration of 50 ng/ml and non-activated cells (healthy cells) were treated with Rhodamine-loaded (Rho, a fluorescent dye) TPM-DNPs, PM-DNPs and Fibroblast (3T3) (ATCC) membrane-coated NPs (3T3-NPs) and allowed to incubate with the cells for 30 minutes at 37° C. 3T3 cell membrane was used as the control group to determine any non-specific uptake. After the incubation period, the cells were washed with PBS and fixed using 4% formaldehyde for 15 minutes and imaged using inverted fluorescence microscopy as described in Matoba, Tetsuya, et al., Journal of cardiology 70.3 (2017): 206-211.

For spectrophotometric analysis quantification of NP uptake, cells were exposed to different concentrations (100 μg/mL, 250 μg/mL, 500 μg/mL, 750 μg/mL) of various NPs for 30 minutes. Subsequently, the cells were washed with 1×PBS and lysed using 1×RIPA (250 μl/well, approximately 30 minutes incubation). The cell extracts were then analyzed for protein contents using the Pierce BCA protein assay kit (Thermo Scientific, Rockford, IL) to determine total cell protein concentration. The fluorescence intensity of Rhodamine emitted from the particles taken up inside the cells was measured using a UV-Vis spectrophotometer. To quantify nanoparticle uptake, the fluorescence intensity of Rho in the lysate was normalized to the total protein concentration in each sample. This normalization method ensures that the uptake measurements reflect the amount of nanoparticles taken up per cell, accounting for differences in cell numbers or protein contents across samples.

### Cell Uptake Studies Under Stress and Flow Conditions

Many cell types are surrounded by moving fluids including vascular endothelial cells that form the inner layer of blood vessels. This liquid flow causes shear stress, a mechanical force that influences the cell morphology and behaviors in many ways. In many standard in vitro experiments, cells are cultured without flow. Under these static conditions, shear stress-dependent cellular changes cannot be considered. In contrast, in vitro cell culture under flow simulates this mechanical mechanism and induces more physiological, in vivo-like behaviors. A syringe pump system for the simulation of physiological systems with defined shear stress would mimic blood vessels, whereas endothelial cells are exposed to the blood flow.

To investigate the effects of shear stress/flow on cellular uptake of membrane coated Cy-7 loaded NPs by activated ECs, cells were seeded onto an 8 μm slide chamber (ibidi, Fitchburg, Wisconsin) at a density of 10⁵ cells/cm². Upon reaching confluence, cells on glass slides were exposed to either 1 or 4 dyne/cm² of media containing nanoparticles at concentration of 100 μg/ml for 30 minutes using the parallel plate flow chamber system as described in Nguyen, Kytai Truong, et al., Annals of biomedical engineering 29 (2001): 145-152. The parallel flow chamber was chosen over other flow systems because of its ability to produce constant levels of shear stress. After washing the cells twice, the chamber was imaged using fluorescent imaging echo microscope. The cells were activated after they were attached to the ibidi chamber and the treatment groups such as NT, TPM-DNPs, PM-DNPs and DNPs were treated and subsequent washes and treatment with nuclei stain (Nucblue, invitrogen) was imaged in fluorescence microscope.

### Cytocompatibility Studies

Cell toxicity and hemocompatibility of TPM-DNPs and PM-DNPs were determined by incubating ECs and heparin-anticoagulated human blood with DINPs at various concentrations (0, 50, 100, 200, and 500 μg/ml), respectively. On cell toxicity, cell viability was studied for 1 day via MTS assays following the manufacturer's instructions and as generally described in Yaman, S. et al., Bioactive Materials 2024, 34, 422-435.

### Cell Proliferation, Migration, and Tube Formation Studies

For cell proliferation, confluent ECs were put in an activated state by incubating them with TNF-α for 2 hours. These activated cells were incubated with no treatment as a negative control, VEGF (25 ng/ml), EPO (20 ng/ml) DNPs, PM-DNPs, and TPM-DNPs at low serum media. At predetermined times (1, 3, and 5 days), cell proliferation was quantified by using MTS assays as described in Perkov, S., Paro, M. M. K., Vidjak, V., & Flegar-Meštrić, Z. (2013). Angiogenesis in Peripheral Arterial Disease. Current Trends in Atherogenesis, 97.

For cell migration, confluent ECs seeded on a 96-well plate were put in an activated state by incubating them with TNF-α for 2 hours. These activated cells were scratched, using a pipette tip to create a wound as described in Zippusch, Sarah, et al., Regenerative Biomaterials 8.5 (2021): rbab039. The cells were then washed with PBS and incubated with no treatment as a negative control, VEGF (25 ng/ml), EPO (20 ng/ml), DNPs, PM-DNPs, and TPM-DNPs in low serum media for 14 hours at 37° C. in normal conditions. A phase contrast microscope was used to obtain the initial and final wound distances. Measurements of the recovered distance were analyzed using Image J analysis software.

For tube formation, in vitro capillary tube formation of activated ECs on a matrix gel was used. Activated ECs with various treatment groups (VEGF, DNPs, PM-DNPs, TPM-DNPs and no treatment group) was seeded on Matrigel in low serum media and incubated for 12 hr. Phase-contrast images will be observed at a 12-hour time-point to show a significant induction of angiogenesis in treatment groups and was analyzed using Image J analysis software.

### In Vitro Uptake Studies of TPM-DNPs

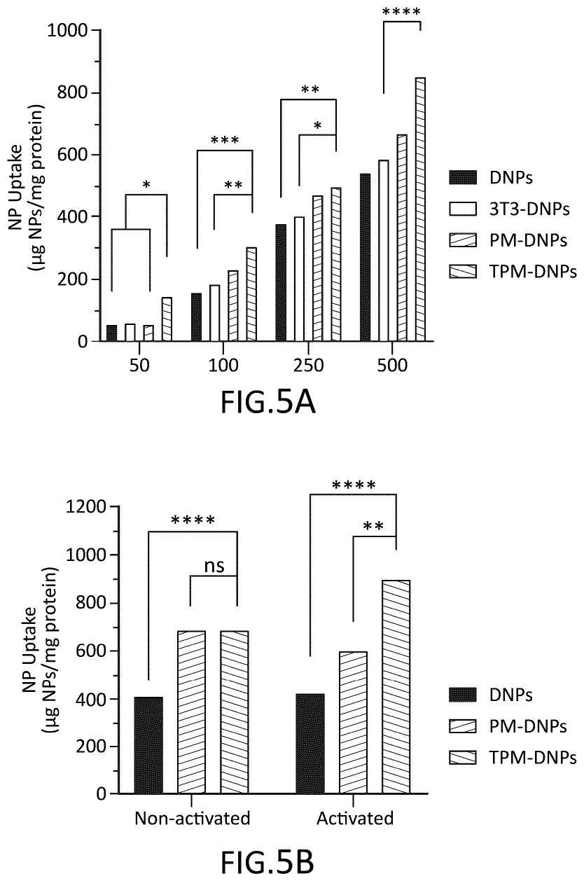

Mouse ECs were utilized to examine cell uptake of TPM-DNPs in comparison to 3T3-DNPs, PM-DNPs, and DNPs from a nanoparticle concentration of 0 to 500 g/mL (FIG. 5A) Fibroblast membrane (3T3) coated NPs served as control to ensure there is no non-specific uptake of NPs by ECs. Rhodamine-loaded TPM-DNPs, PM-DNPs, 3T3-DNPs and DNPs were used in this study. ECs (80k cells/well) were seeded on a 24 well plate overnight. Cells were put into a quiescent state for 2 hours in TNF-α media before being incubated another 2 hours in different concentrations of TPM-DNPs (0, 100, 250, and 500 μg/ml). After 4 hours, fluorescent images were taken to trace TPM-DNPs (Rhodamine) inside ECs using Echo Revolve fluorescent microscope. Images were then processed using ImageJ software for brightness and contrast adjustment. The results showed EC uptake was increased when the concentration of TPM-DNPs was higher. Microscopic views showed more particles of DNPs taken up by ECs compared to the control group (ECs without NP incubation) and 3T3 DNPs (p<0.05). As the concentration increased, the cell uptake of nanoparticles increased significantly, and it was found to be highest in the PM-DNPs and TPM-DNPs and statistically significant uptake was seen at all concentrations for TPM-DNPs when compared to 3T3-DNPs (p<0.05).

Since the inflammatory condition of ECs resembles the ischemic site of PAD, the ECs were activated using 50 ng/ml of tumor necrosis factor-alpha (TNF-α), and cellular uptake studies were performed using PM-DNPs and TPM-DNPs at 100 μg/mL concentration. After 4 hours of treatment, the uptake was found to be more in TPM-DNPs than the PM-DNPs which is consistent with the theory that TNF-α triggered inflammation that helps uptake of TPM-DNPs more than the PM-DNPs (p<0.05) (FIG. 5B). These studies demonstrated the targeting capability of these TPM-DNPs.

### Cytocompatibility and Cell Proliferation

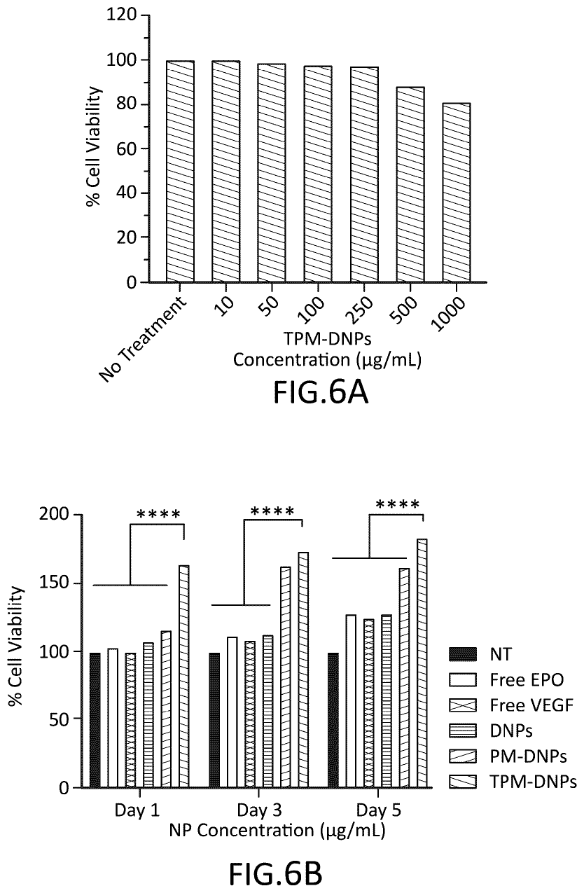

The in-vitro cytotoxicity of blank TPM-DNPs was analyzed by MTS assays. ECs were seeded at 10,000 cells/well in 96-well plates and incubated in 37° C., 5% CO₂ incubator for 24 hrs. Cells were incubated with the nanoparticles at various concentrations (from 0 to 1 mg/mL) in complete media for 48 hrs. The cells were cyto-compatible at a concentration up to 250 ug/mL; however, cell viability was reduced at concentrations of 500 and 1000 μg/mL (p<0.05) (FIG. 6A, which shows cyto-compatibility analysis of TPM-DNPs on ECs to determine its in vitro cytotoxic effect on different concentrations (0 ug/mL-1000 μg/mL). Cell viability was quantified via MTS assay (n=4), ***P<0.05). ECs were treated with TNF-α to stimulate an inflammatory condition. Cells were seeded onto 96 well plates at confluent density overnight. Cells were washed and replenished with TPM-DNPs in low serum media for 1 day. Then cell viability was analyzed via MTS assays (n=4). No treatment (cells in low serum media) served as a negative control. The results demonstrated 80% of the cells were viable at a concentration of 1 mg/mL under stress conditions.). The cell viability of all concentrations is still above 80%, which is considered as cyto-compatible materials for clinical uses.

The therapeutic efficacy of agents released from DNPs was assessed in ECs. ECs were treated with various groups such as no treatment as a negative control, VEGF as positive control (25 ng/mL), EPO as positive control (20 ng/mL), EPO loaded PLGA (DNPs), PM-DNPs and TPM-DNPs. For cell proliferation studies, after activating the cells using TNF-α, the cells were incubated with the various groups in low serum media, cell viability over time (day 1, 3, and 5) was quantified using MTS assays. TPM-DNPs treated cells significantly proliferated the ECs under stimulated conditions when compared to VEGF, EPO, PM-DNPs, and no treatment, especially Day 5 (FIG. 6B, which shows cell-viability analysis of TPM-DNPs on ECs. Cells were seeded onto 96 well plates at seeding density overnight. Cells were washed and replenished with TNF-α in low serum media. Then cells were treated with free EPO, free VEGF, DNPs, PM-DNPs and TPM-DNPs and viability was analyzed via MTS assays for Day 1, 3 and 5. No treatment (cells in low serum media) served as a negative control). Cell viability was better with DNPs, PM-DNPs and TPM-DNPs compared to that of VEGF and significantly better compared to that of non-treatment (p<0.05). The results were normalized to no treatment group and TPM-DNPs had more than 25% increase in cell growth when compared to other groups at the end of Day 5.

### In Vitro Therapeutic Effects of TPM-DNPs on ECs

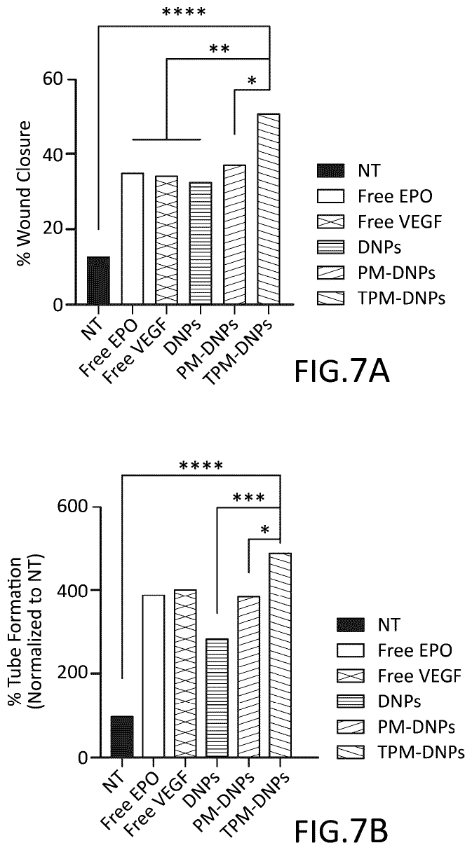

To determine the in vitro therapeutic effects of TPM-DNPs, activated ECs were used to investigate the cell migratory and tube formation properties of these NPs. 100% wound closure refers to complete cell migration leading to closure of the wound, indicative of curability. DNPs, PM-DNPs, TPM-DNPs, free VEGF and EPO showed significant reduction in the initial wound gap (FIG. 7A, which shows cell migration of activated ECs in 2% serum media treated with treatment groups free EPO, free VEGF, DNPs, PM-DNPs and TPM-DNPs). The targeting ability of TPM-DNPs boosted cell uptake of NPs, which in turn resulted in significantly higher migration (p<0.05) of cells compared to VEGF, EPO, and other groups. There was about a 32% increase in cell migration when compared to VEGF (FIG. 7A). TPM-DNPs showed the highest EC migration when compared to no treatment and positive control groups: VEGF and EPO. Within 12 hours, the percentage tube length of TPM-DNPs was significantly higher than that of DNPs, PM-DNPs and NT. The TPM-DNPs efficacy for tube formation was a little higher compared to positive controls, VEGF and EPO (FIG. 7B, which shows activated ECs in low serum media seeded on matrigel and treated with free EPO, free VEGF, DNPs, PM-DNPs and TPM-DNPs for 12 hours (n=3)). ECs under stressed conditions facilitated statistically significant (p<0.05) angiogenesis of about 30% when compared to those of the controls. These results suggest the enhanced angiogenesis potential of the TPM-DNPs for treatment, particularly of PAD.

### Cell Uptake of TPM-DNPs Under Physiological Flow Conditions

The effect of cell uptake when treated Cy7 dye loaded NP conjugated with transfected membrane (TPM-DNPs) under flow was evaluated with syringe pump connected to an Ibidi chamber coupled to a pump to simulate shear stresses (4.00 dyne/cm²) as would be found in vivo. Cellular uptake of TPM-DNPs was greater than those of other control groups, indicating that activation of cell with TNF-α induced more expression of ICAM1 receptors on cell surfaces, and the targeted NPs were binding more at the inflamed cells compared to those of non-targeted groups under flow conditions mimicking the physiological state of the body.

### Discussion

To investigate the targeting capability of TPM-DNPs, a cell uptake study was performed, where it was observed that TPM-DNPs at each concentration showed statistically higher uptake when compared to 3T3-DNPs. To evaluate the targeting ability of TPM-DNPs, both TPM-DNPs and PM-DNPs were treated on activated and non-activated ECs to mimic the physiological conditions and in the activated ECs. The targeting ability of TPM-DNPs was more prominent than PM-DNPs since the fluorescent intensity measured for TPM-DNPs was statistically higher than PM-DNPs. This also confirms the ICAM1 specific targeting and uptake of TPM-DNPs due to the presence of TPM-DNPs.

To determine TPM-DNPs releasing EPO protein for inducing in vitro therapeutic effects, mice ECs were used for cell proliferation, migration and tube formation studies since they are cell lines derived from mice endothelial cells. Many angiogenic factors increase endothelial cell numbers by enhancing both proliferation and survival.

The cell viability was performed for 5 days, and cell viability was significantly better when cells were exposed to TPM-DNPs compared to that of the control groups. Free EPO and VEGF were observed to be less viable than TPM-DNPs. Without being bound by theory, this could be because the cells were washed after 2 hours, after treating with therapeutic agents and/or NPs to mimic the physiological conditions; the washing step would have washed away the free proteins but since TPM-DNPs were up taken by the cells, the effects of EPO protein on ECs were seen in the form of an increase in cell viability. Migratory and tube formation studies were performed to evaluate the main function of TPM-DNPs.

The results demonstrated significant wound healing properties of ECs under stressful conditions and facilitated angiogenesis in a nutrient deprived environment compared to controls.

### Example 4

### Evaluation of Engineered Pericyte Cell Membrane Coated Nanoparticles In Vivo.

In vivo targeting efficacy evaluation of TPM-DNPs on PAD created balb/c mice, and in vivo near infrared imaging studies were done on these mice. Studies were performed to compare the TPM-DNPs group and control groups (PM-DNPs and saline). In addition, both intramuscular and intravenous injections of TPM-DNPs in PAD mouse models were performed to see the retention of NPs on gastrocnemius muscles and other organs. For in vivo images, fluorescence intensities from muscles on PAD created legs were measured. Ex vivo organ imaging and fluorescent accumulation were also determined to evaluate biodistribution of TPM-DNPs. At the end point of the studies, spectrophotometric analysis of homogenized organs was also done to confirm the findings of in vivo and ex vivo image analysis. NP distributions of all organs and muscles for both types of injection were plotted and analyzed to reveal the effect of TPM membrane coating on PLGA NPs. Followed by biodistribution studies, therapeutic evaluation of TPM-DNPs was performed. The blood flow ratio of the ischemic limb (left)/non-ischemic limb (right) was measured and determined using a laser doppler perfusion imager (Perimed). At various time points post treatment, all mice were challenged with an acute exercise (maximal endurance) as previously described. The maximal running distances and number of stimulations were recorded. At the end of the time point, histological evaluation of the muscle tissue was achieved by staining with hematoxylin and eosin (H & E), Trichrome and different cell surface markers for neovascularization, proliferation, stem cell recruitment and inflammatory markers were used to examine muscle degeneration and tissue fibrosis in the ischemic regions as described by Abd El Aziz, M. T., et al., Journal of advanced research 6.2 (2015): 133-144.

### Material and Method

### Creation Of PAD Mice (Animal Model Creation)

A mouse model of unilateral hindlimb ischemia was created as described Lin, Jenny B., et al., International journal of molecular sciences 16.5 (2015): 11131-11177 with an approved IACUC animal protocol A21.003. Aging is an important factor responsible for impaired angiogenesis in PADs, and blood perfusion of ischemic hindlimbs in old mice was significantly decreased compared to young mice after surgery. Briefly, 12-month-old-BALB/C mice (age relevant to the old age of human) of both sexes bought from Charles River Laboratory were anesthetized with isoflurane in oxygen. Mice underwent surgery following approved IACUC protocols, including subcutaneous Buprenorphine SR administration and anesthesia with 2% isoflurane in oxygen. Post-surgery, blood perfusion confirmed ischemia before mice were returned to recovery cages. The left femoral artery and its branches were ligated to generate hindlimb ischemia based on the procedures described in Padgett, Michael E., et al., Journal of visualized experiments: JoVE 112 (2016): 54166. The right hindlimb was kept intact as a control non-ischemic limb. Animals were randomly selected to different treatment and control groups at the fourth post-surgery day. The model creation was confirmed through Hematoxylin and eosin (H&E) staining, Masson's Trichrom staining (Statlab) and Laser Speckle Contrast Imaging (LSCI, PeriCam PSI NR system, Perimed, California). The right hindlimb was kept intact as a control non-ischemic limb. Animals were randomly selected to different treatment and control groups at the fourth post-surgery day in any animal studies later. The model creation was confirmed through Hematoxylin and eosin (H&E) staining and Masson's Trichrom staining (Statlab) and Laser Speckle Contrast Imaging (LSCI, PeriCam PSI NR system, Perimed, California).

Bio-Distribution Study of Cy 5 TPM-DNPs (IM Vs. IV)

The biodistribution study examined whether intravenously or intramuscularly injected TPM-DNPs targeted gastrocnemius muscle on PAD mice. About 2 mg of Cy5-loaded TPM-DNPs and PM-DNPs were resuspended in 150 μl saline. The membrane was dyed with DiA (ThermoFisher) for imaging purposes. These NPs were intravenously (IV) and intramuscularly (IM) injected along the ischemic gastrocnemius muscle on PAD mice for these bio-distribution studies. Saline served as a control group. Biodistribution of Cy5-loaded NPs was assessed by an in vivo fluorescent imaging system (Kodak in vivo Fx Pro system, Carestream Molecular Imaging, Connecticut) via whole animal in vivo imaging and ex vivo organ imaging. At the 2 hour-timepoint, 4 mice/group were sacrificed to collect various organs including liver, kidneys, spleen, and gastrocnemius muscles (injured and healthy legs) for ex vivo imaging as described in Lin, Jenny B., et al. The tissues were further homogenized by Precellys Evolution Homogenizers (Bertin Instruments, Maryland), and fluorescent intensities of lysed samples were determined via spectrophotometer (Tecan Infinite M200 Spectrophotometer, Tecan, California). Quantification of TPM-DNPs distribution was determined as the amount of NPs per gram of tissue. The PAD muscle leg tissue sections for IV and IM injections were also imaged using a confocal microscope with a z thickness of 8 μm using DiA fluorescence and DAPI (nuclear stain) channel.

### Investigation of the In Vivo Effectiveness of TPM-DNPs on PAD Mice

Mice received three different doses of TPM-DNPs (1 mg, 2 mg, and 3 mg per mouse) to determine the optimal therapeutic dose. Treatment efficacy was assessed through blood perfusion and physical function tests. The optimal dose was then selected for further evaluation in vivo. For optimal dose studies, animals were divided into five groups: sham (saline), VEGF (positive control), free EPO, PM-DNPs, and TPM-DNPs. Treatments were administered intravenously to PAD mice, and outcomes were evaluated weekly using LCSI imaging and treadmill (Panlab Touchscreen Treadmill, Harvad Apparatus, Holliston, MA) endurance tests. The treadmill protocol began at 6 m/min and increased by 2 m/min every 2 minutes for 12 minutes, then maintained at 18 m/min. Exhaustion was defined as the mouse spending more than 5 seconds on the shock grid without resuming treadmill activity. Animals were sacrificed at 7-, 14- and 21-days post-treatment for histological analysis following the treatment plan (Post surgery, baseline readings for LSCI and treadmill were taken. Followed by IV injection, there were the treatment groups saline, VEGF, free EPO, PM-DNPs and TPM-DNPs. The LSCI and treadmill readings were taken on weeks 1, 2 and 3. Post 3rd week, the animals were sacrificed, and the organs were collected for IHC staining.).

### Evaluation of Histological Staining

Gastrocnemius muscles were collected, sectioned, stained, and analyzed by H&E, trichrome and immunohistology staining. Muscle sections in paraffin (5 μm thick) were H&E stained to assess structure and inflammation. Immunohistology used fluorescent-labeled secondary antibodies (Abcam protocol): antigens retrieved varied by marker (CD31, CD34, EPOR, Ki67) as described in Park, In Su, et al., The Anatomical Record: Advances in Integrative Anatomy and Evolutionary Biology 296.1 (2013): 168-177. Primary antibodies incubated overnight at 4° C., followed by PBS washes and 1-hour incubation with FITC-labeled secondaries. DAPI counterstained nuclei before mounting; ECHO Revolve microscope captured fluorescent images. CD31 antibodies quantified ECs for angiogenesis; EPOR and CD34 antibodies assessed EPOR expression and EPC recruitment respectively (refer to S. Elliott, et al., Biologics 6 (2012) 163-189 and T. Annese, et al., Exp Cell Res 374 (2) (2019) 266-273.

### Statistical Analysis

GraphPad Prism 8 (GraphPad Software Inc., San Diego, USA) was used to perform statistical analysis. One-way ANOVA with Dunnett multiple comparisons and Tukey's multiple comparisons tests were done for all analyses. Triplicate samples were used for all the studies if not specified.

### PAD Model Creation and Evaluation

The PAD mouse models were established by surgically ligating the left femoral artery and its branches, following a documented procedure [I. H. Jeong, et al., Cell Death Dis 11 (8) (2020) 624 and M. E. Padgett, et al, J Vis Exp (112) (2016). 10.3791/54166]. PAD models were created on Balb/c mice aged 6-8 months, and left leg femoral artery was ligated. This procedure is commonly used to induce ischemia in experimental settings (In this model, the left femoral artery is carefully isolated and ligated using surgical sutures, thereby occluding blood flow to the lower limb. The ligation induces ischemia in the distal leg, mimicking the reduced blood flow seen in PAD patients. The procedure is typically followed by monitoring functional recovery and vascular remodeling, with assessments such as laser Doppler imaging or histological analysis used to evaluate the extent of tissue ischemia and revascularization. This femoral artery ligation model serves as a widely used tool for studying the pathophysiology of PAD and evaluating potential therapeutic interventions.).

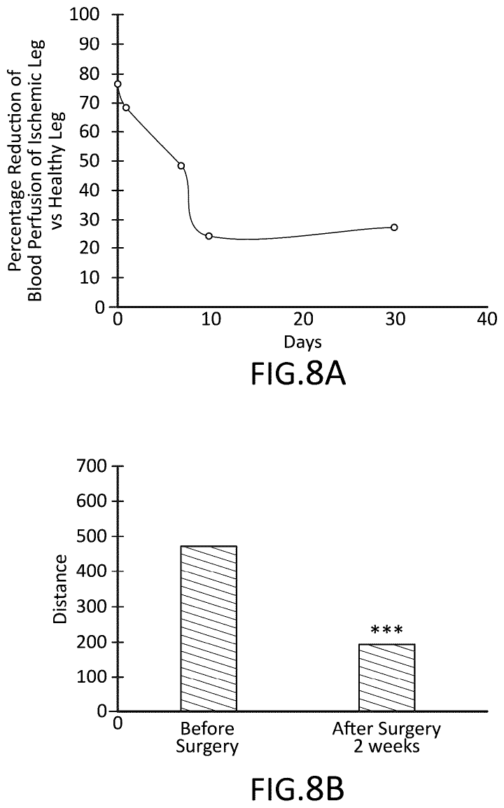

To assess the extent of ischemia, laser speckle contrast imaging (LSCI) was utilized. LSCI provides visual and quantitative measurements of blood perfusion. The experimental LSCI images revealed significantly lower blood perfusion in the ischemic leg compared to the healthy leg (non-surgery leg of same mice) (FIG. 8A, showing a graphical representation of blood perfusion data showing the significant reduction of blood flow in PAD leg when compared to normal/healthy leg.). The blood perfusion was reduced to about 55% when compared to a healthy leg. The distance travelled by mice after surgery significantly dropped by 50% when compared to those of healthy (FIG. 8B, showing a comparison of physical activity, as represented by distance traveled on treadmill according to a PAD model between healthy and ischemia induced mice.).

Once the model was created, the mice were euthanized and the Gastrocnemius muscles from both PAD leg and control leg tissues were extracted, embedded and sectioned into 7 μm slices. These tissues were stained with Masson's Trichrome and H&E to evaluate the tissues at the pathological level. These findings underscore the reliability of the PAD model in studying ischemic conditions. Trichrome staining of muscle tissue of PAD mice had significantly higher deposition of collagen when compared to healthy mice. Hemoxylin and eosin staining of paraffin embedded muscle tissues indicated high density of fibrotic tissues and inflammation and showed tissue injury through inflammatory cells and cell disruption. No significant tissue damage was seen in the healthy muscle group. These results coincide with the literature where the PAD model on mice demonstrated reduction in blood flow and endurance, whereas the tissue was fibrous.

### Bio-Distribution of Cy5-Loaded TPM-DNPs on PAD Mice

Retention of NPs in mouse hind legs (injured vs. control) and distribution of NPs to other organs over 2 hours following intravenous (IV) and intramuscular (IM) injections of Cyanine5 (Cy 5)-loaded membrane coated PLGA nanoparticles were compared. To determine the best administration routes for PAD treatment, a biodistribution study was performed to investigate IM injection and IV injection in PAD mice. The investigated mice were sacrificed after 3 hours for ex vivo imaging to evaluate NPs that remained at the ischemic tissues as well as accumulated in organs. For IV injection, the fluorescent imaging of the mice and ex-vivo imaging showed that the accumulation of NPs for TPM-DNPs was more than for PM-DNPs. There were some NPs observed in the kidney, liver, and spleen but the fluorescent intensities of PM-DNPs in these organs were greater than TPM-DNPs. There was some accumulation of NPs observed in the heart and lung for the PM-DNPs treatment group. Despite NPs retention observed in different organs, there was a significant number of NPs accumulated in the PAD muscles for TPM-DNPs when compared to the non-targeted group PM-DNPs. All the intensities were normalized for organ auto fluorescence of the saline group. The results showed retention of NPs in muscle and that NPs remained at ischemic tissues for IM injection. There were fluorescent signals observed in other organs also.

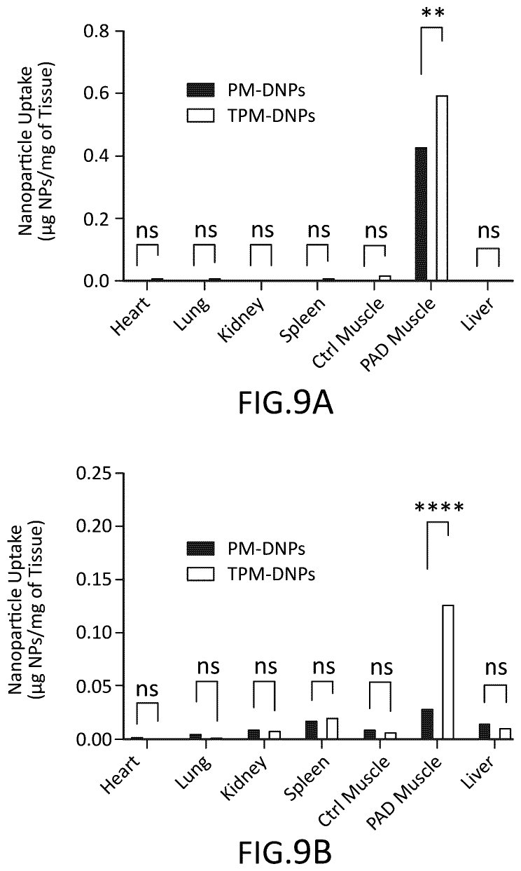

For IM injection, the fluorescent imaging of the mice and ex-vivo imaging showed similar accumulation of NPs for both TPM-DNPs and PM-DNPs. There were some NPs retained in the kidney and liver but the fluorescent intensities of both TPM-DNPs and PM-DNPs in these organs were similar to the NP retention in kidney and liver as previously seen. To quantify the amounts of NPs on each tissue, all organs were weighed, homogenized, and measured for fluorescent intensities using a spectrophotometer (FIG. 9A shows the analyzed data of IM injection of Cy5 TPM-DNPs were distributed in homogenized tissues; and FIG. 9B shows the analyzed data of IV injection of Cy5 TPM-DNPs were distributed in homogenized tissues. Quantified dye loaded NPs accumulated in tissues and organs using UV spectrophotometer. The tissues were weighed and homogenized, and the supernatant was measured for Cy5 fluorescence at 649/667 nm excitation/emission. The supernatant was also measured for protein content of the cells via BCA assay.). Some organ tissues were used for creating sections for confocal imaging to view the in-depth penetration of NPs and uniform distribution of NPs in the muscle tissues. The spectrophotometer results calculated as μg of NPs uptake per mg of tissue weight showed that the accumulation of NPs in PAD muscles for IM injection was highest when compared to IV.

The fluorescent intensity for PAD muscle for groups TPM-DNPs and PM-DNPs in IV injection were similar, but for IM injection, there was a significant difference in the fluorescent intensities between TPM-DNPs and PM-DNPs (FIGS. 9A-B). The targeted NPs (TPM-DNPs) showed better accumulation of NPs when compared to PM-DNPs for IV injection. TPM-DNPs and PM-DNPs had similar intensities for IM injection, suggesting that the targeting moiety had no role or a lesser role in accumulating at the ischemic sites for IM. Hence, to evaluate if the NPs got accumulated at the capillary level or just diffused at the injection site for (IM), and to determine if the NPs were distributed evenly throughout the tissue or were concentrated at one site, confocal imaging of the tissue from the site of injection was collected. The data showed the NPs accumulate more on the gastrocnemius muscles when they are injected intramuscularly, especially at the injection sites. The TPM-DNPs had statistically higher accumulation on the muscles than PM-DNPs for IV injection. These NPs were also uniformly distributed throughout the gastrocnemius muscles for IV injection compared to those of IM injection.

### Therapeutic Effects of TPM-DNPs in LSCI and Treadmill Running.

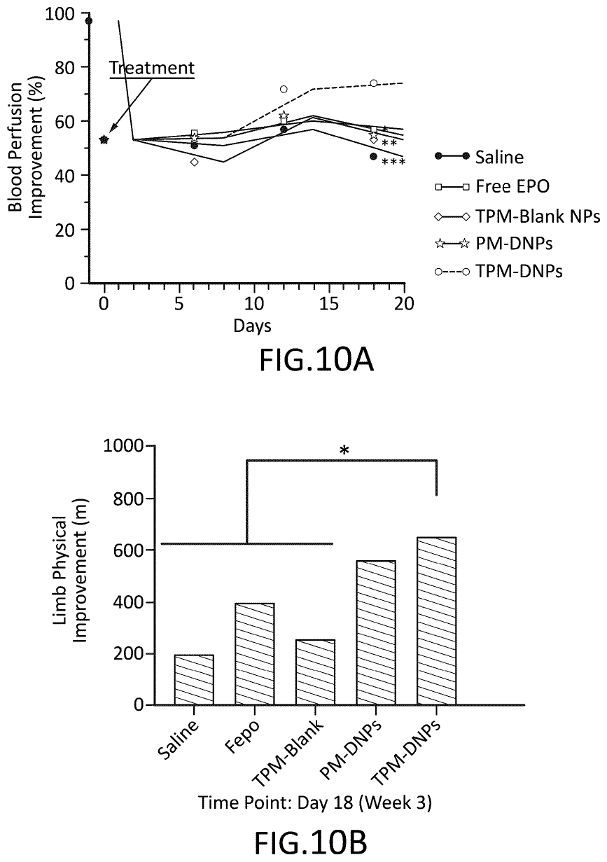

To evaluate the in vivo therapeutic effects of TPM-DNPs, 150 μL of each treatment was administered intravenously into the left gastrocnemius muscle (the injured leg) of Balb/c mice (n=4 per group) following surgery. The study involved five treatment groups: saline (sham, control), free EPO (1000 U/kg of mice), TPM-blank, PM-DNPs and TPM-DNPs (amount was normalized to drug release data and free EPO). The normal leg on the right side of each mouse served as a control for normalizing the blood perfusion ratio (ischemic leg/normal leg ratio). Blood perfusion was assessed using LSCI on day 0, week 1, week 2 and week 3 to monitor changes over time. The results showed that TPM-DNPs significantly improved blood perfusion in the injured hindlimb compared to the sham, or uninjured limb group (FIG. 10A). In contrast, all mice in the control group developed necrotic toes and/or limbs within 1-2 weeks. TPM-DNPs statistically increased physical limb improvement (endurance in m) when compared to free EPO, TPM-blank and saline when plotted for the time point-day 18 (week 3) (FIG. 10B).

### Histology Staining

Ischemic muscles were gently dissected from the tibia bone and fixed with 4% paraformaldehyde overnight before embedded in the paraffin for tissue sections. Histological analysis of ischemic hindlimb tissues was conducted to further confirm the therapeutic efficacy of TPM-DNPs and assess immune responses. Tissue damage due to ischemia and inflammatory cell infiltration were evident in the sham and empty vector NP groups. In addition, muscles treated with TPM-DNPs exhibited more extensive vascular networks compared to the saline group. TPM-DNPs demonstrated better preservation of muscle tissue integrity and formation of vascular networks as well as more connecting tissues and less fibrotic tissues when compared to the control saline group. TPM-DNPs displayed notable therapeutic benefits by significantly enhancing vascular structure formation and reducing inflammatory cell recruitment at the injury site.

For immunohistochemical staining, tissue sections were analyzed using antibodies against anti-CD31 (an endothelial cell marker), anti-CD34 (an endothelial progenitor cell marker), anti-Ki67 (proliferating marker), anti-EPOR (EPOR expression) and anti-ICAM1 (inflammatory marker) as described in the literature (A. A. Tadbir, et al Asian Pac J Cancer Prev 13 (10) (2012) 10.7314/apjcp.2012.13.10.5155 5155-5159; Z. Yang, et al., Am J Pathol 184 (4) (2014) 10.1016/j.ajpath.2013.12.023 1230-1239; K. Habas, Tissue Cell 54 (2018) 10.1016/j.tice.2018.09.002 139-143.) The staining protocol followed the double staining immunohistochemistry procedure detailed on the Abcam website. A single secondary antibody conjugated with FITC was used for all immunostaining of CD31, CD34, Ki67, EPOR and ICAM1 markers, allowing visualization of positive cells.

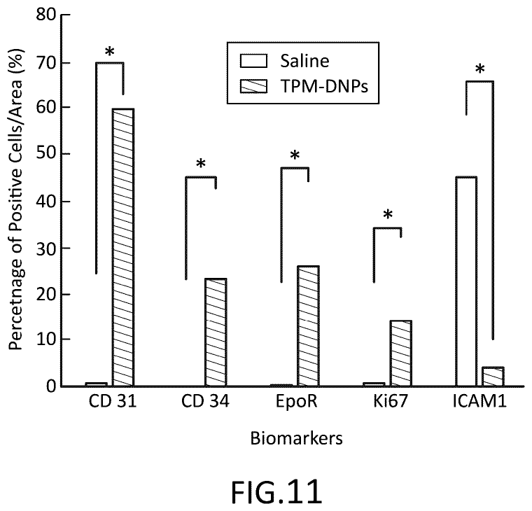

The results revealed that mice treated with TPM-DNPs exhibited a significant increase in the number of cells positive for CD31, CD34, and Ki67. The histology staining also showed high EPOR protein expression and less inflammation when compared to that of saline. Quantitative analysis demonstrated that TPM-DNPs enhanced capillary density by 60% when compared to the saline group. (FIG. 11, showing TPM-DNPs significantly decreased the number of cells positive with ICAM1. Data are presented as Mean±SD for percentage of various biomarkers positive per field of view. Student's and Welch's t-test were run and * indicated significant difference (P<0.01) with respect to corresponding saline groups.) There was a significant increase in EPC recruitment in the treatment group, which was 76 times higher than in the saline group. EPOR and Ki67 expression was significantly elevated, being 88 and 18 times greater than in the saline group, respectively. The inflammatory marker ICAM1 decreased significantly in TPM-DNPs when compared to saline group.

### Discussion

For the biodistribution study, TPM-DNPs had statistically higher accumulation on the muscles than PM-DNPs for IV injection. Without being bound by theory, this could have been due to the targeting moiety presented on the surface of the membrane coated NPs. Since there is no targeting aspect in PM-DNPs, the accumulation was statistically less when compared to TPM-DNPs. The targeting ability of NPs in vivo was also confirmed by Ungerleider et al., wherein significant accumulation of NPs through IV injection was shown. However, the accumulation of TPM-DNPs was similar to that of PM-DNPs for IM injection. Without being bound by theory, this could have been because the NPs were directly injected into the muscle, and targeting aspects of TPM-DNPs did not have much of a role in accumulation, which was also confirmed through spectrophotometer data. Spectrophotometric analysis of homogenized organs showed a similar trend of both in vivo and ex vivo imaging measurements. For IM injection in the liver and spleen, there was a lower accumulation of membrane coated groups, when compared to IV injection. In the kidney, heart and lungs, PM-DNPs showed higher accumulation than that of the TPM-DNPs for IV injection. This also indicates that due to the ICAM1 binding ligand coated NPs, there was specific binding of TPM-DNPs in the capillaries while circulating in ischemic site when compared to PM-DNPs which had non-specific binding which would be cleared off by the system. Both ex-vivo and spectrophotometer data confirm that PM-DNPs also accumulated at the ischemic site indicating the recruitment of pericyte like cells (NPs in this case) in both types of injections. Without being bound by theory, pericytes have stem cell-like properties that cause their recruitment and accumulation at inflammatory sites for both types of injections.

The confocal results provide evidence that although the accumulation of TPM-DNPs via IV injection is comparatively less than IM injection, it was evenly distributed at the capillary level. The NPs via IM injection was localized at the point of the injection site, and it cannot diffuse longitudinally. On the other hand, the IV confocal imaging data suggests the uniform distribution of NPs along the muscle. This also suggests that although there could be less accumulation of NPs on the ischemic site, it would be uniform throughout the muscle at the cellular level and the effect of the NPs loaded with drug in combination with increase in bioavailability of these NPs due to presence of targeting moiety would help in increasing the therapeutic efficacy via IV injection mode. The uniformity of the biodistribution of drug plays an important role in inducing angiogenesis and recruiting stem cells to rebuild the injured tissue. Hence, for therapeutic evaluation of TPM-DNPs, IV injection site was chosen. There is no evidence in the literature wherein the researchers have compared IM vs IV modes of injection to evaluate the targeting capability. Disclosed herein is an autologous system where the hybrid biofunctionalized and biomimetic cell membrane system specific to each patient can serve as a universal drug carrier that can load any drug and can be used as a nanocarrier system for different cardiovascular diseases.

TPM-DNPs enhanced physical activity when compared to control groups. The free EPO protein group resulted in blood perfusion levels similar to the sham group. Without being bound by theory, this is likely due to the rapid clearance of the protein by the immune system and instability of the drug due to the short half-life of the protein. Mice treated with PM-DNPs displayed similar blood perfusion compared to the saline and free EPO groups, but less than those treated with TPM-DNPs, which might be due to less bioavailability and bioaccumulation of NPs. Without being bound by theory, due to non-specific targeting, PM-DNPs might have been cleared more quickly from the system. Treadmill endurance tests revealed that mice receiving TPM-DNPs showed superior physical recovery compared to PM-DNPs, free EPO, and saline groups (FIG. 10B) on week 3. These results indicate that intravenous injections of TPM-DNPs accumulated at the ischemic sites due to specificity for ICAM1 targeting and that the engineered membrane coated NPs prolonged the retention of therapeutic TPM-DNPs, enhancing their availability and therapeutic effects. TPM-DNPs significantly improved blood perfusion and physical functions in the injured legs of PAD mice, suggesting a promising potential for similar efficacy in humans.

### EMBODIMENTS

Additional exemplary embodiments contemplated herein are as follows:

Embodiment 1. A composition comprising a nanoparticle defining an interior volume and an exterior surface; a therapeutic agent disposed within the interior volume of the nanoparticle; and a cellular membrane coating disposed on or encapsulating the exterior surface of the nanoparticle; wherein the cellular membrane coating comprises an ICAM1 binding ligand.

Embodiment 2. The composition of Embodiment 1, wherein the nanoparticle is a polymeric nanoparticle, an inorganic nanoparticle or a lipid-based nanoparticle.

Embodiment 3. The composition of Embodiment 1 or 2, wherein the nanoparticle is a polymeric nanoparticle and comprises poly(lactic-co-glycolic acid), polylactic acid, polyglycolic acid, polyglutamic acid, polycaprolactone, polylysine or mixtures thereof.

Embodiment 4. The composition of any of Embodiments 1-3, wherein the therapeutic agent comprises small molecule therapeutics, regenerative factors, protein peptides and/or plasmids for gene therapy or combinations thereof.

Embodiment 5. The composition of any of Embodiments 1-4, wherein the therapeutic agent comprises vascular endothelial growth factor, fibroblast growth factor, hepatocyte growth factor, stromal derived growth factor, platelet derived growth factor, erythropoietin.

Embodiment 6. The composition of any of Embodiments 1-5, wherein the therapeutic agent comprises peripheral blood-derived or bone marrow-derived stem cells, mesenchymal stem cells, or marker-specific subsets of bone marrow cells with angiogenic properties, or the therapeutic agent comprises peptides, antibodies, and/or aptamers that recognize specific targets associated with PAD pathology.

Embodiment 7. The composition of any of Embodiments 1-6, wherein the cellular membrane is derived from a pericyte.

Embodiment 8. The composition of any of Embodiments 1-7, wherein the pericyte cellular membrane expresses the ICAM1 binding ligand.

Embodiment 9. The composition of any of Embodiments 1-8, wherein the ICAM1 binding ligand comprises an integrin, fibrinogen, or hyaluronan, or a binding fragment of any of the foregoing.

Embodiment 10. The composition of any of Embodiments 1-9, wherein the ICAM1 binding ligand comprises LFA-1 or Mac 1, or a binding fragment of any of the foregoing.

Embodiment 11. A pharmaceutical composition comprising an effective amount of the composition of any of Embodiments 1-10 and a pharmaceutically acceptable carrier or excipient.

Embodiment 12. The pharmaceutical composition of Embodiment 11, wherein the nanoparticle is a polymeric nanoparticle; and the cellular membrane is derived from a pericyte.

Embodiment 13. The pharmaceutical composition of any of Embodiments 11-12, wherein the ICAM1 binding ligand is an integrin, fibrinogen, or hyaluronan, or a binding fragment of any of the foregoing.

Embodiment 14. The pharmaceutical composition of any of Embodiments 11-13, wherein the ICAM1 binding ligand comprises LFA-1 or Mac 1, or a binding fragment of either of the foregoing.

Embodiment 15. A method of treating a condition of a patient in need thereof, the method comprising: disposing the composition of any of Embodiments 1-10 within a biological compartment of the patient.

Embodiment 16. The method of Embodiment 15, wherein the condition is peripheral artery disease.

Embodiment 17. The method of Embodiment 15 or 16, wherein the nanoparticle is a polymeric nanoparticle; and the cellular membrane is derived from a pericyte.

Embodiment 18. The method of any of Embodiments 15-17, wherein the therapeutic agent is erythropoietin.

Embodiment 19. The method of any of Embodiments 15-18, wherein the ICAM1 binding ligand is an integrin, fibrinogen, or hyaluronan, or a binding fragment of any of the foregoing.

Embodiment 20. The method of any of Embodiments 15-19 wherein the composition of any of Embodiments 1-14 engages with an endothelial cell at a PAD disease site.

Embodiment 21. A method of manufacturing a membrane-coated nanoparticle, wherein a therapeutic agent is disposed within the interior volume of the nanoparticle; a cellular membrane coating extracted from a pericyte is disposed on or encapsulating the exterior surface of the nanoparticle; and the nanoparticle comprises a polymer; the method comprising obtaining a nanoparticle comprising a polymer; loading the nanoparticle with a therapeutic agent; coating a cellular membrane on the nanoparticle, wherein the cellular membrane comprises an ICAM1 binding ligand.

Embodiment 22. The method of Embodiment 21, further comprising engineering the pericyte to express an ICAM1 binding ligand on its membrane.

All patent documents referred to herein are incorporated by reference in their entireties. Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. A composition comprising:
a nanoparticle defining an interior volume and an exterior surface;
a therapeutic agent disposed within the interior volume of the nanoparticle; and
a cellular membrane coating disposed on or encapsulating the exterior surface of the nanoparticle;
wherein the cellular membrane coating comprises an ICAM1 binding ligand.

2. The composition of claim 1, wherein the nanoparticle is a polymeric nanoparticle, an inorganic nanoparticle or a lipid-based nanoparticle.

3. The composition of claim 2, wherein the nanoparticle is a polymeric nanoparticle and comprises poly(lactic-co-glycolic acid), polylactic acid, polyglycolic acid, polyglutamic acid, polycaprolactone, polylysine or mixtures thereof.

4. The composition of claim 1, wherein the therapeutic agent comprises a small molecule therapeutic, a regenerative factors, a protein peptide and/or a plasmid for gene therapy or combinations thereof.

5. The composition of claim 1, wherein the therapeutic agent comprises vascular endothelial growth factor, fibroblast growth factor, hepatocyte growth factor, stromal derived growth factor, platelet derived growth factor, erythropoietin or combinations thereof.

6. The composition of claim 1, wherein the therapeutic agent comprises peripheral blood-derived or bone marrow-derived stem cells, mesenchymal stem cells or marker-specific subsets of bone marrow cells with angiogenic properties, or the therapeutic agent comprises peptides, antibodies, and/or aptamers that recognize specific targets associated with PAD pathology.

7. The composition of claim 1, wherein the cellular membrane is derived from a pericyte.

8. The composition of claim 7, wherein the pericyte cellular membrane is engineered to express the ICAM1 binding ligand.

9. The composition of claim 1, wherein the ICAM1 binding ligand comprises an integrin, fibrinogen, or hyaluronan, or a binding fragment of any of the foregoing.

10. The composition of claim 9, wherein the ICAM1 binding ligand comprises LFA-1 or Mac-1, or a binding fragment of either of the foregoing.

11. A pharmaceutical composition comprising an effective amount of the composition of claim 1 and a pharmaceutically acceptable carrier or excipient.

12. The pharmaceutical composition of 11, wherein
the nanoparticle is a polymeric nanoparticle; and
the cellular membrane is derived from a pericyte.

13. The pharmaceutical composition of claim 12, wherein the ICAM1 binding ligand is an integrin, fibrinogen, or hyaluronan, or a binding fragment of any of the foregoing.

14. The pharmaceutical composition of claim 13, wherein the ICAM1 binding ligand comprises LFA-1 or Mac-1, or a binding fragment of either of the foregoing.

15. A method of treating a condition of a patient in need thereof, the method comprising:
disposing the composition of claim 1 within a biological compartment of the patient.

16. The method of claim 15, wherein the condition is peripheral artery disease.

17. The method of claim 16, wherein
the nanoparticle is a polymeric nanoparticle; and
the cellular membrane is derived from a pericyte.

18. The method of claim 16, wherein the therapeutic agent is erythropoietin.

19. The method of claim 17 wherein the ICAM1 binding ligand is an integrin, fibrinogen, or hyaluronan, or a binding fragment of any of the foregoing.

20. A method of manufacturing a membrane-coated nanoparticle,
wherein a therapeutic agent is disposed within the interior volume of the nanoparticle;
a cellular membrane coating extracted from a pericyte is disposed on or encapsulating the exterior surface of the nanoparticle; and
the nanoparticle comprises a polymer;
the method comprising
obtaining a nanoparticle comprising a polymer;
loading the nanoparticle with a therapeutic agent;
coating a cellular membrane on the nanoparticle, wherein the cellular membrane comprises an ICAM1 binding ligand.
